(19) United States
(12) Patent Application Publication
(10) Pub. No.: US 2010/0081613 A1
(43) Pub. Date: Apr. 1, 2010

(54) METHODS AND COMPOSITIONS FOR ENHANCING MEMORY

(75) Inventors: Ottavio Arancio, New York, NY (US); Daniela Puzzo, Catania (IT); Cristina Alberini, New York, NY (US); Paul Mathews, Irvington, NY (US)

Correspondence Address: CADWALADER, WICKERSHAM & TAFT LLP, ONE WORLD FINANCIAL CENTER, NEW YORK, NY, 10281, US

(73) Assignee: THE TRUSTEES OF COLUMBIA UNIVERSITY IN THE CITY OF NEW YORK, New York, NY (US)

(21) Appl. No.: 12/414,160

(22) Filed: Mar. 30, 2009

Related U.S. Application Data
(63) Continuation of application No. 12/185,396, filed on Aug. 4, 2008, now abandoned.
Application No. 12/185,396 is a continuation of application No. 11/870,724, filed on Oct. 11, 2007, now abandoned.
(60) Provisional application No. 60/850,734, filed on Oct. 11, 2006.

(51) Int. Cl. A61K 38/16; A61P 25/00; A61P 25/28; A61P 25/16

(52) U.S. Cl. 514/12

(57) ABSTRACT

The invention is directed to methods for enhancing memory by administering low doses of beta amyloid peptide. The invention also encompasses methods for increasing synaptic plasticity in a subject which comprises administering to the subject low doses of beta amyloid peptide.

This application is a continuation of U.S. application Ser. No. 12/185,396, which was filed on Aug. 4, 2008, which is a Continuation of U.S. application Ser. No. 11/870,724, which was filed on Oct. 11, 2007, which claims priority to U.S. Provisional Application No. 60/850,734 filed on Oct. 11, 2006. These applications are hereby incorporated by reference in their entireties.

The invention disclosed herein was made with U.S. Government support under NIH Grant No. NS49442 from the NINDS to Ottavio Arancio; NIH Grant No. RO1 MH65635 from the NIMH to Cristina Alberini; and NIH Grant No. R21NS45357-02 from the NINDS to Paul Mathews. Accordingly, the U.S. Government may have certain rights in this invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### BACKGROUND OF THE INVENTION

Alzheimer's disease (AD) amyloid plaques consist of aggregated polypeptides of ˜39-42 amino acids, which are now known as amyloid-β (Aβ) peptides. Abnormally high levels of Aβ have been shown to cause toxic effects such as synaptic dysfunction and memory loss. Synaptic alterations are highly correlated with the severity of clinical dementia. Indeed, AD begins as a disorder of synaptic function leading to cognitive deficits and neurodegenerative pathology. However, Aβ is also produced at low levels in the brain of normal healthy subjects, including humans, in which interstitial fluid Aβ levels are regulated by synaptic activity through synaptic vesicle exocytosis. The in vivo concentration in the rodent brain has been estimated to be in the range of 200-1000 pM, with Aβ₄₂ at the lower end of this range and Aβ₄₀ at the higher.

Drug discovery efforts are ongoing to develop strategies to decrease Aβ load by the use of agents that inhibit the β- or γ-secretases (or increase α-secretase activity and thus decrease Aβ production), by the use of drugs that inhibit Aβ oligomerization, or by the use of treatments, such as Aβ immunization, that appear to augment the removal of Aβ from the brain. However, it is important to better define and understand the physiological role of Aβ, because it may play an important role when designing effective and safe approaches to treating neurological conditions such as memory disorders or Alzheimer's Disease.

### SUMMARY OF THE INVENTION

The present invention provides the discovery that administration of Aβ₄₂ at a concentration approximately equal to its physiological levels (200 pM) enhances both long-term potentiation (LTP), a widely studied type of synaptic plasticity that is thought to be associated with learning and memory [15], and both contextual fear memory and inhibitory avoidance (IA), two types of fear conditioning-based type of memory in rodents. The invention provides for methods that enhance synaptic plasticity and memory wherein the method comprises administering to a subject's brain an effective amount of amyloid beta peptide, such as Aβ₄₂. Additionally, the invention provides evidence that acute, antibody-mediated depletion of the endogenously produced Aβ dramatically interferes with LTP in vitro and fear memory formation in vivo. Based on these strong, collaborative findings, the invention provides Aβ itself is a critical positive-modulator of memory at physiological concentrations within the normal CNS.

The invention provides a method for enhancing memory of a subject, the method comprising administering to the subject an amount of an amyloid beta peptide wherein the amount of amyloid beta peptide administered is sufficient to achieve a concentration of about 200 pM in the hippocampal tissue of the subject. The invention also provides for a method for enhancing synaptic plasticity in neurons of a subject, the method comprising administering to the subject a low dose of a beta amyloid peptide. In one embodiment, the amyloid beta peptide is Aβ₄₂ having SEQ ID NO: 1. In another embodiment, the amyloid beta peptide is a peptide with at least about 75% identity to SEQ ID NO:1, or at least about 80% identity to SEQ ID NO:1, or at least about 85% identity to SEQ ID NO:1, or at least about 90% identity to SEQ ID NO:1, or at least about 95% identity to SEQ ID NO:1, or at least about 97% identity to SEQ ID NO:1, or at least about 99% identity to SEQ ID NO:1.

In one aspect of the invention, the amount of beta amyloid peptide in the brain following administration is from about 125 pM to about 500 pM, or from about 130 pM to about 480 pM, or from about 140 pM to about 475 pM, or from about 150 pM to about 450 pM, or from about 160 pM to about 440 pM, or from about 170 pM to about 430 pM, or from about 180 pM to about 420 pM, or from about 190 pM to about 410 pM, or from about 200 pM to about 400 pM, or from about 210 pM to about 350 pM, or from about 200 pM to about 300 pM, or from about 200 pM to about 225 pM, or from about 200 pM to about 250 pM, or from about 200 pM to about 275 pM. The invention provides for methods wherein the subject is suffering from Alzheimer's Disease, head trauma, or an attention deficit disorder. The invention also provides for methods wherein the subject is suffering from a memory disorder. In one aspect of the invention, the memory disorder comprises or is associated with Alzheimer's disease, Parkinson's disease, Pick's disease, a Lewy body disease, amyotrophic lateral sclerosis, Huntington's disease, Creutzfeld-Jakob disease, Down syndrome, multiple system atrophy, neuronal degeneration with brain iron accumulation type I (Hallervorden-Spatz disease), pure autonomic failure, REM sleep behavior disorder, mild cognitive impairment (MCI), cerebral amyloid angiopathy (CAA), mild cognitive deficits, aging, vascular dementias mixed with Alzheimer's disease, a neurodegenerative disease characterized by abnormal amyloid deposition, or any combination thereof. In another aspect of the invention, the amyloid beta peptide is administered to the brain of the subject via intralesional, intraperitoneal, intramuscular or intravenous injection; by infusion; by liposome-mediated delivery; or topical, nasal, oral, anal, ocular or otic delivery, or any combination thereof. In another aspect of the invention, the amyloid beta peptide is a peptidomimetic.

### BRIEF DESCRIPTION OF THE FIGURES

FIG. 1 is a histogram showing levels of endogenous Aβ₄₂ and Aβ₄₀ detected by sandwich ELISA from rat cerebellum, cortex, and hippocampus (mean±SE, n=3).

FIG. 2 shows a Tris-Tricine PAGE Western blot analysis of Aβ samples (monomeric, oligomeric and do-decameric) in non-denaturing/non-reducing conditions. Monomeric Aβ (lane 1) produced a blotted band around 4.5 kDa size while oligomeric Aβ was visible at about 9 kDa size (dimers) or 14 kDa size (trimers) (lane 2). Dodecameric Aβ was primarily detected at ˜54 kDa (lane 3). Each Aβ form was prepared as described.

FIG. 3 shows a Western blot analysis demonstrating that monoclonal antibody m3.2 specifically detects rodent APP metabolites, including Aβ, and fails to interact with human APP or Aβ.

FIG. 4 shows a cartoon of APP holoprotein, APP metabolites (sAPP, CTF, Aβ) and monoclonal antibodies. This cartoon diagrams the cleavage sites of APP holoprotein during its proteolytic processing and the anti-APP/APP metabolite monoclonal antibodies, all of which react with the rodent protein/fragments.

FIGS. 5A-5E: Human Aβ₄₂ rescues reduction of LTP and fear memory induced by an anti-rodent Aβ monoclonal antibody (m3.2 Ab). (A) Perfusion of hippocampal slices with m3.2 mAb (2 μg/ml)—but not anti-human-specific PS1 antibodies that do not bind to any rodent proteins (control mAb, 2 μg/ml)—for 20 min prior to a θ-burst stimulation blocks LTP. The antibodies do not affect baseline transmission. The horizontal bar indicates the period during which the antibodies were added to the bath solution. The three arrows indicate the θ-burst in this and the other graphs. (B) Perfusion of hippocampal slices with human Aβ₄₂ (200 pM)—but not scramble Aβ42 (200 pM)—plus m3.2 mAb (2 μg/ml) for 20 min prior to a θ-burst stimulation, rescues LTP without affecting baseline transmission. The horizontal bars indicate the period during which antibodies and Aβ were added to the bath solution. (C) Schematic representation showing cannulas implanted bilaterally into the dorsal hippocampi. (D) Bilateral injections of m3.2 mAb (1 μg in 1 μl)—but not control mAb (1 μg in 1 μl)—into dorsal hippocampi, 15 min prior to training, worsens contextual conditioning performance as the mice are exposed to the context after 24 hrs. (E) Bilateral injections of human Aβ₄₂ (200 pM)—but not scramble Aβ₄₂ (200 pM)—plus m3.2 mAb (1 μg in 1 μl) into dorsal hippocampi, 15 min prior to training, re-establishes normal contextual conditioning performance as the mice are exposed to the context after 24 hrs.

FIGS. 6A-6C: Aβ₄₂ at a physiologically relevant concentration enhances LTP and associative memory (A) Perfusion of hippocampal slices with a preparation containing human Aβ₄₂ (200 pM)—but not scramble Aβ₄₂ (200 pM)—for 20 min prior to a theta-burst stimulation increases the amounts of LTP. Aβ₄₂ does not affect baseline transmission. The horizontal bar indicates the period during which Aβ was added to the bath solution. (B) Bilateral injections of human Aβ₄₂ (200 pM)—but not scramble Aβ₄₂ (200 pM)—into dorsal hippocampi, 15 min prior to training, enhances contextual conditioning performance as the mice are exposed to the context after 24 hrs. (C) Concentration-response curve for the effect of Aβ₄₂ on LTP indicating that the peptide has its maximal enhancing effect at 200 pM, whereas it has an opposite detrimental effect above 20 nM. The dotted line corresponds to the amount of potentiation in vehicle-treated slices. The residual potentiation was calculated by averaging the last 5 minutes of LTP.

FIGS. 7A-7B: Aβ₄₂ at a physiologically relevant concentration enhances PTP without affecting AMPA receptor currents. (A) Perfusion of hippocampal slices with human Aβ₄₂ (200 pM) does not affect amplitude of AMPA currents (B) Perfusion of hippocampal slices with human Aβ₄₂ (200 pM) in the presence of D-APV (50 μM) enhances PTP. The horizontal bars indicate the period during which Aβ and/or APV were added to the bath solution.

FIGS. 8A-8D: Aβ₄₂ at a physiologically relevant concentration enhances synaptic plasticity and fear memory through α7-nAchRs. (A) Perfusion of hippocampal slices with MCL (3 μM) concurrent with human Aβ₄₂ (200 pM) and D-APV (50 μM) blocks the PTP enhancement. The horizontal bars indicate the period during which MCL, Aβ and APV were added to the bath solution. (B) Perfusion of hippocampal slices with α-BgTx (0.1 μM) concurrent with human Aβ₄₂ (200 pM) and D-APV (50 μM) blocks the PTP enhancement. The enhancement is still present after wash-out of α-BgTx if slices are perfused again with Aβ₄₂ (200 pM). The horizontal bars indicate the period during which MCL, Aβ and APV were added to the bath solution. (C) Perfusion of hippocampal slices with human Aβ₄₂ (200 pM) for 20 min prior to a theta-burst stimulation does not increase the amounts of LTP in slices from α7-KO mice, whereas it still enhances potentiation in slices from WT littermates. The horizontal bar indicates the period during which Aβ was added to the bath solution. (D) Bilateral injections of human Aβ₄₂ (200 pM) into dorsal hippocampi, 15 min prior to training, does not enhance contextual conditioning performance as α7-KO mice are exposed to the context after 24 hrs, whereas it still enhances freezing in WT littermates.

FIG. 9: Aβ₄₂ at a physiologically relevant concentration produces a dramatic increase in LTP in APP-KO mice. Perfusion of hippocampal slices with human Aβ₄₂ (200 pM) for 20 min prior to a theta-burst stimulation produces a dramatic increase in the amounts of LTP in slices from APP-KO mice. The increase in LTP is still present in Aβ-treated slices from WT littermates. APP-KO slices treated with vehicle show a reduction in LTP compared to vehicle-treated WT slices. The horizontal bar indicates the period during which Aβ was added to the bath solution.

FIGS. 10A-10B: Cued conditioning is not affected by m3.2 mAbs. (A) Bilateral injections of m3.2 mAb (1 μg in 1 μl) or control mAb (1 μg in 1 μl) into dorsal hippocampi, 15 min prior to training, do not affect freezing during cued conditioning. (B) Bilateral injections of human Aβ₄₂ (200 pM) or scramble Aβ₄₂ (200 pM) concurrent with m3.2 mAb (1 μg in 1 μl) into dorsal hippocampi, 15 min prior to training, do not affect freezing during cued conditioning.

FIG. 11: Post-tetanic application of Aβ₄₂ at a physiologically relevant concentration does not vary LTP. Perfusion of hippocampal slices with human Aβ₄₂ (200 pM) for 20 min after a theta-burst stimulation does not affect LTP, nor baseline transmission. The horizontal bar indicates the period during which Aβ₄₂ was added to the bath solution.

FIG. 12: Cued conditioning is not affected by human Aβ₄₂. Bilateral injections of human Aβ₄₂ (200 pM) or scramble Aβ₄₂ (200 pM) into dorsal hippocampi, 15 min prior to training, do not affect freezing during cued conditioning.

FIGS. 13A-13B: Block of α7-nAchRs does not affect PTP. (A) Perfusion of hippocampal slices with MCL (3 pM) in D-APV (50 μM) does not affect PTP. The horizontal bars indicate the period during which MCL and APV were added to the bath solution. (B) Perfusion of hippocampal slices with α-BgTx (0.1 pM) in D-APV (50 pM) does not affect PTP. The horizontal bars indicate the period during which MCL and APV were added to the bath solution.

FIGS. 14A-14B: Basal synaptic transmission is normal in α7- and APP-KO mice. (A) Input-output curves showing similar basal synaptic transmission in α7-KO and WT-mice. Similar results were obtained when the fEPSP slope was plotted versus the amplitude of the fiber afferent volley. (B) Input-output curves showing similar basal synaptic transmission in APP-KO and WT-mice. Similar results were obtained when the fEPSP slope was plotted versus the amplitude of the fiber afferent volley.

FIGS. 15A-15C: (A) Mean latency of acquision (Acq) and memory retention (Lat) of groups of rats trained in inhibitor avoidance, and, bilaterally injected into the hippocampi with either m3.2 mAb or control mAb 15 min before training. Memory retention was tested 24 hrs after training. (B) Mean latency of Acq and Lat of groups of rats trained in inhibitor avoidance, and, bilaterally injected into the hippocampi with either m3.2 mAb plus Aβ₄₂ (200 pM) or scramble Aβ₄₂ (200 pM) as well as control mAβ plus Aβ₄₂ (200 pM) or scramble Aβ₄₂ (200 pM) 15 min before training. Memory retention was tested 24 hrs after training. (C) Mean latency of Acq and Lat of groups of rats trained in inhibitor avoidance, and, bilaterally injected into the hippocampi with Aβ₄₂ (200 pM), scramble Aβ₄₂ (200 pM) or vehicle. Memory retention was tested 24 hrs after training.

FIG. 16 shows a bar graph showing mean latency of memory retention of groups of rats trained in IA, and, immediately after training, bilaterally injected into the hippocampi with either m3.2 antibody, or control monoclonal antibody. Memory retention was tested 24 hours after training.

FIG. 17: Perfusion with m3.2 mAb (2 μg/ml) for 20 minutes after tetanization did not affect LTP (214.10±27.72 in m3.2 mAb treated slices, n=5; vs 234.70±20.00 in vehicle treated slices, n=4; F(1,7)=0.22, p=0.647). The horizontal bar indicates the period during which the antibodies were added to the bath solution.

FIG. 18: The effect of Aβ₄₂ at 200 pM is specific for this peptide as perfusion with Aβ₄₀ (200 pM) for 20 minutes before tetanization did not enhance LTP (215.02±25.45 vs 205.28±16.69% in vehicle-treated slices; two-way ANOVA F(1,15)=0.22, p=0.633), whereas Aβ₄₀ (200 nM) caused an impairment of LTP (123.44±10.43, two-way ANOVA F(1,13)=10.82, p=0.007 compared to vehicle treated slices). Perfusion with Aβ₄₀ either at 200 pM or at 200 nM had no effect on baseline transmission (91.25±4.05 vs 98.82±1.84%, respectively). N indicates the number of slices per condition. The horizontal bar indicates the period during which Aβ₄₀ was added to the bath solution.

FIG. 19: Perfusion of the slices with 200 pM Aβ₄₂ for 20 min produced a reduction in paired pulse facilitation (PPF) at the CA3-CA1 connection in hippocampus (20 ms: 103.85±11.00% of facilitation in Aβ₄₂-treated slices vs 140.48±17.33% in vehicle treated slices, t(34)=1.85, p=0.073; 30 ms: 126.67±10.50% of facilitation vs 166.83±20.61%), t(34)=1.81, p=0.078; 40 ms: 148.23±9.44% of facilitation vs 186.76±18.33%, t(34)=1.95, p=0.059; 50 ms: 159.66±9.53% of facilitation vs 202.44±25.46%, t(34)=1.65, p=0.107), suggesting that Aβ modifies presynaptic mechanisms of short-term plasticity linked to PPF (PPF occurs as two stimuli are closely spaced with each other. It is thought to reflect build-up of residual Ca²⁺ due to the action potential from the previous depolarization of the terminal, leading to enhanced transmitter release at the arrival of the second stimulus. As the time between stimuli increases, facilitation approaches zero, since they are seen as independent (Zucker, 1989)). This effect was dependent upon nicotinic receptor activation as the non-selective nAchR blocker mecamylamine (MCL) (3 μM for 20 min) re-established normal facilitation (20 ms: 138.18±22.01% of facilitation, t(34)=0.09, p=0.924; 30 ms: 158.58±16.79% of facilitation, t(34)=0.34, p=0.763; 40 ms: 176.71±17.51% of facilitation, t(34)=0.43, p=0.665; 50 ms: 197.49±22.30% of facilitation, t(34)=0.16, p=0.869). These experiments were performed in the same slices (n=18) in the following manner: at first we measured the amounts of PPF in the presence of vehicle, then we added Aβ₄₂, finally we added Aβ₄₂ plus MCL.

FIG. 20: Perfusion of the slices with MCL (3 μM for 20 min) did not affect PPF (214.53±20.85% with MCL vs 217.62±29.48% with vehicle; t(18)=0.08, p=0.933). These experiments were performed in the same slices (n=10) in the following manner: at first we measured the amounts of PPF in the presence of vehicle, and then we added MCL.

FIG. 21: Perfusion of the slices with m3.2 mAb (2 μg/ml) for 20 minutes produces a slight increase of PPF at 20 and 30 msec intervals (173.27±15.34% and 189.06±12.30), (t(10)=1.65, p=0.128, t(10)=1.88, p=0.088 for the 2 intervals)), but not at the higher intervals. The effect of m3.2 mAbs was rescued by 200 pM Aβ₄₂ concurrent with the m3.2 mAb perfusion for 20 min (122.10±11.17% and 147.27±9.81% at 20 ms and 30 ms) (t(10)=0.44, p=0.667, t(10)=0.18, p=0.0855 for the 2 intervals compared to vehicle alone). These data indicate that the block of endogenous Aβ modifies presynaptic mechanisms of facilitation at brief intervals corresponding to high frequency stimulation applied to produce LTP. These experiments were performed in the same slices (n=6) in the following manner: at first we measured the amounts of PPF in the presence of vehicle, then we added m3.2 mAbs, finally we added Aβ₄₂ plus m3.2 mAb.

FIG. 22: Perfusion of hippocampal slices with m3.2 mAbs (2 μg/ml) for 20 minutes concurrent with D-APV (50 μM) reduces the amounts of PTP (71% of PTP in the presence of m3.2 mAbs compared to vehicle, t(12)=2.26, p=0.043, n=7). The m3.2 mAb reduction in PTP was rescued by Aβ₄₂ (200 pM for 20 min) concurrent with m3.2 mAbs plus D-APV (96% of PTP before m3.2 mAb perfusion, t(12)=0.27, p=0.790 compared to vehicle alone). These data are consistent with the reduction and rescue in LTP following perfusion with m3.2 mAbs first and then with m3.2 mAbs plus Aβ shown in FIG. 21. They support the hypothesis that the block of endogenous Aβ impairs neurotransmitter release during the tetanus. The horizontal bars indicate the period during which m3.2 mAbs, Aβ and APV were added to the bath solution.

### DETAILED DESCRIPTION OF THE INVENTION

The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference.

The invention is directed to methods for enhancing or improving memory where the methods comprise administering a low dose of a beta amyloid peptide to the subject in an amount to increase synaptic plasticity in the neurons of the subject and thereby enhance or improve memory of the subject.

The following abbreviations are used:

TABLE 1
Abbreviations

| | |
|---|---|
| aa | amino acid |
| Acq | mean latency of acquisition |
| AD | Alzheimer's disease |
| Aβ | amyloid-β |
| Ab | amyloid-β |
| A-beta | amyloid-β |
| APP | amyloid-β precursor protein |
| sAPPα | soluble APP, α-cleaved |
| sAPPβ | soluble APP, β-cleaved |
| α-BgTx | α-bungarotoxin |
| control mAb | anti-human-specific, |
| | intracellular epitope PS1 |
| | monoclonal antibody, NT1 |
| CTF | carboxy-terminal fragment |
| αCTF | C-terminal membrane |
| | associated APP fragment, α- |
| | cleaved |
| βCTF | C-terminal membrane |
| | associated APP fragment, β- |
| | cleaved |
| CNS | central nervous system |
| DH | dounce homogenate |
| FAD | familial Alzheimer's disease |
| fEPSP | field excitatory post-synaptic |
| | potential |
| IA | inhibitory avoidance |
| Lat | mean latency of memory |
| | retention |
| LTP | long-term potentiation |
| m3.2 mAb | anti-rodent Aβ monoclonal |
| | antibody |
| MCL | mecamylamine |
| nAchR | nicotinic acetylcholine |
| | receptors |
| NMDA | N-methyl D-aspartate |
| PS | presenilin |
| SB | sucrose buffer |
| SEC | size-exclusion |
| | chromatography |
| SEM | standard error of the mean |
| wt | wild-type |

Amyloid Beta

As used herein, “amyloid beta peptide” as used herein encompasses any amyloid beta peptide. For example, an Aβ peptide termed Aβ₄₂ is an amyloid beta peptide having the amino acid sequences as follows, derived from amyloid-precursor protein: DAEFRHDSGYEVHHQKLVFFAEDVGSNKGAIIGLMVGGVVIA (SEQ ID NO:1) (Homo sapiens: NP_{—}000475.1, AAW82435.1, AAB59502.1, NP958816.1, NP958817.1, CAA31830.1; Pan troglodytes, AAW74286.1; Macaca fascicularis: BAE01907.1; Canis familiaris: AAR97728.1; Gallus gallus: NP_{—}989639.1, AAG00593.1; Sus scrofa NP 999537.1, ABB82034.1; Chelydra serpentina serpentine, AAN04908.1; Oryctolagus cuniculus, CAA39594.1; Ovis sp., CAA39595.1, Bos Taurus, CAA39589.1, Cavia sp., CAA39591.1). DAEFGHDSGFEVRHQKLVFFAEDVGSNKGAIIGLMVGGVVIA (SEQ ID NO:2) (Mus musculus: NP_{—}031497.2; AAH70409.1, AAP23169.1; Rattus norvegicus: AAH62082.1; Rattus rattus, CAA30488.1; Cricetulus griseus, AAB86608.1). DAEFRHDSGYEVHHQKLVFFAEDMSSNKGAIIGLMVGGIVIA (SEQ ID NO:3) (Antechinus stuartii, CAJ31109.1). DSEYRHDTAYEVHHQKLVFFAEEVGSNKGAIIGLMVGGVVIA (SEQ ID NO:4) (Xenopus laevis, AAH70668.1; African clawed frog, JH0773). For other sequences and relative ID numbers see the NCBI GenBank.

In one embodiment, the peptide is a peptidomimetic. The invention provides for an isolated peptide comprising an amino acid sequence that is about 75% or less, about 80% or less, about 85% or less, about 90% or less, about 95% or less, about 96% or less, about 97% or less, about 98% or less or about 99% or less identical to the amino acid sequences of SEQ ID NOS:1-5. In one embodiment, the peptide is linked to a carrier. In another embodiment, the peptide is a peptide that includes KLVFFAE (SEQ ID NO:5) (the central sequence of amyloid beta 42) and about 15-25 other amino acid residues. Other variants that might mimic Aβ action are sold by American Peptide (Sunnyvale, Calif., USA) such as: Aβ1-11 (DAEFRHDSGYE (SEQ ID NO:6)), Aβ1-28 (DAEFRHDSGYEVHHQKLVFFAEDVGSNK (SEQ ID NO:7)), Aβ1-38 (DAEFRHDSGYEVHHQKLVFFAE DVGSNKGAIIGLMVGG (SEQ ID NO:8)), Aβ1-40 (DAEFRHDSGYEVHHQKLVFFAEDVGSNKGAIIGLMVGGVV (SEQ ID NO:9)), Aβ10-20 (YEVHHQKLVFF (SEQ ID NO:10)), Aβ12-28 (VHHQKLVFFAEDVGSNK (SEQ ID NO:11)), Aβ22-35 (EDVGSNKGAIIGLM (SEQ ID NO:12)), Aβ25-35 (GSNKGAIIGLM (SEQ ID NO:13)), Aβ31-35 (IIGLM (SEQ ID NO:14)), Aβ32-35 (IGLM (SEQ ID NO:15)), A BRI Peptide (1-34) (ASNCFAIRHFENKFAVETLICSRTVKKNIIEEN (SEQ ID NO:16)), Aβ (1-40), amide (DAEFRHDSGYEVHHQKLVFFAEDVGSNKGAIIGLMVGGVV-NH₂ (SEQ ID NO:17)), Aβ (1-43) (DAEFRHDSGYEVHHQKLVFFAEDVGSNKGAIIGLMVGGVVIAT (SEQ ID NO:18)), Aβ (17-28) (LVFFAEDVGSNK (SEQ ID NO:19)), Aβ (33-42) (GLMVGGVVIA (SEQ ID NO:20)), (Glp3) Aβ (3-42) (Glp-FRHDSGYEVHHQKLVFFAEDVGSNKGAIIGLMVGGVVIA (SEQ ID NO:21)), Amyloid BRI Protein (1-23) (EASNCFAIRHFENKFAVETLICS (SEQ ID NO:22)), Amyloid BRI Protein (1-34) (reduced) (ASNCFAIRHFENKFAVETLICSRTVKKNIIEEN (SEQ ID NO:23)), Amyloid Dan Protein (1-34) (ASNCFAIRHFENKFAVETLICFNLFLNSQEKHY (SEQ ID NO:24)), Amyloid Dan Protein (1-34) (reduced) (ASNCFAIRHFENKFAVETLICFNLFLNSQEKHY (SEQ ID NO:25)), Aβ (10-35), amide (YEVHHQKLVFFAEDVGSNKGAIIGLM-NH₂ (SEQ ID NO:26)), Aβ (17-40) (LVFFAEDVGSNKGAIIGLMVGGVV (SEQ ID NO:27)), Biotinyl-β-Amyloid (1-42), Prion Protein (118-135) human (AGAVVGGLGGYMLGSAMS (SEQ ID NO:28)), [Arg13] Aβ (1-40), [Arg22] Aβ (1-40), [Asp22] Aβ (1-40), [Cys] Aβ (1-40), [Gln11] Aβ (1-16), [Gln11] Aβ (1-28), [Gln11] Aβ (1-40), [Gln22] Aβ (1-40), [Gln22] Aβ (6-40), [Gly21] β-Amyloid (1-40), [Gly22] β-Amyloid (1-40), [Lys22] Aβ (1-40), [Nle35] Aβ (1-40), [Phe10] Aβ (1-40).

Amyloid beta (used interchangeably with beta amyloid or Aβ or A-beta or A-b or Abeta) is a protein fragment of 39-43 amino acids that is the main constituent of amyloid plaques in the brains of Alzheimer's disease patients. The progressive deposition of insoluble Aβ in the brain as β-amyloid plaques is a key neuropathological hallmark of AD [16], along with the accumulation of intracellular paired helical filament tau [17]. Similar plaques appear in some variants of Lewy body dementia and in inclusion body myositis, a muscle disease. Aβ also forms aggregates coating cerebral blood vessels in cerebral amyloid angiopathy.

Aβ is formed after sequential cleavage of the amyloid precursor protein (APP) by the β- and γ-secretases. γ-secretase produces Aβ₄₂ if cleavage occurs in the endoplasmic reticulum and Aβ₄₀ if the cleavage is in the trans-Golgi network. APP is a transmembrane glycoprotein. Autosomal-dominant mutations in APP cause hereditary early-onset Alzheimer's disease, likely as a result of altered proteolytic processing. Increases in either total Aβ levels or the relative concentration of both Aβ₄₀ and Aβ₄₂ (where the former is more concentrated in cerebrovascular plaques and the latter in neuritic plaques) have been implicated in the pathogenesis of both familial and sporadic Alzheimer's disease. Due to its more hydrophobic nature, the Aβ₄₂ is the most amyloidogenic form of the peptide. However the central sequence KLVFFAE (SEQ ID NO:5) is known to form amyloid on its own, and probably forms the core of the fibril.

Amyloid peptide and its accumulation in the brain of people with Alzheimer's disease have been widely investigated; yet, the peptide is produced in the brain throughout life in normal individuals. However, it is not known whether amyloid has a physiological role in the brain. It is a discovery of this invention that amyloid is a critical factor in memory in the normal brain. Indeed, and paradoxically, the use of drugs that mimic amyloid structure, or targeted to the receptor(s) on which amyloid acts under normal physiological conditions, or even of amyloid itself or amyloid derivatives can be used in the methods of this invention to enhance memory at appropriate concentrations.

The Aβ used in the methods of this application can be in monomeric form. In another embodiment, the Aβ used in the methods of this invention can be in oligomeric form, including dimers, trimers, tetramers, pentamers, eptamers, decamers, dodecamers or any combination thereof.

Data has been obtained showing that administration of Aβ₄₂ at a concentration approximately equal to its physiological levels enhances long-term potentiation (LTP), a widely studied cellular model of learning and memory, as well as contextual fear learning and inhibitory avoidance (IA), two fear conditioning-based types of memory in rodents.

Data has been obtained showing that depletion of Aβ₄₂ impairs LTP, contextual fear learning and IA.

Memory Enhancement

This invention provides that Aβ itself is a critical positive-modulator of memory at physiological concentrations within the normal CNS. Thus, the invention provides methods for increasing or improving or enhancing memory by administering Aβ to a subject in an appropriate therapeutic amount, over an appropriate time period, and at appropriate intervals during that time period. This approach has the advantage of combining unique expertise and tools: electrophysiology has the ability to dissect the role of Aβ in LTP modulation and the capacity to investigate in vitro the mechanisms by which Aβ acts to modulate LTP; molecular mechanisms of memory consolidation in the intact animal are studied; in vitro findings in the intact CNS are conducted to demonstrate behavioral relevance for the slice-LTP findings; study of APP and Aβ metabolism, and manipulation of the levels of key APP metabolites, including Aβ, in the rodent CNS. The present invention provides methods for administering Aβ at physiological concentrations, so that LTP is enhanced in vitro and both contextual fear learning and IA memory are enhanced in vivo.

The invention provides methods for enhancing memory by administering Aβ, at physiological concentrations to enhance LTP in vitro and both contextual fear learning and IA memory in vivo. LTP, contextual fear learning and IA memory are enhanced by exogenous Aβ₄₂ at physiologically relevant concentrations. This invention provides a role for the endogenous Aβ, produced by the neurons in slices or in the intact brain, in enhancement of LTP as well as in enhancement of contextual fear learning and IA memory. Additionally, the invention provides for use of oligomeric Aβ₄₂ at a concentration of from about 125 pM to about 275 pM, and not Aβ₄₀ at a concentration of about 200 pM, to mediate these effects.

The invention provides the discovery that Aβ, at physiological concentrations in the non-diseased brain, is an important positive-modulator of memory. Electrophysiology was used to dissect the role of A-beta in LTP modulation (see Example 6) and to investigate in vitro the mechanisms by which A-beta acts to modulate LTP (see Examples 7, 8 and 9).

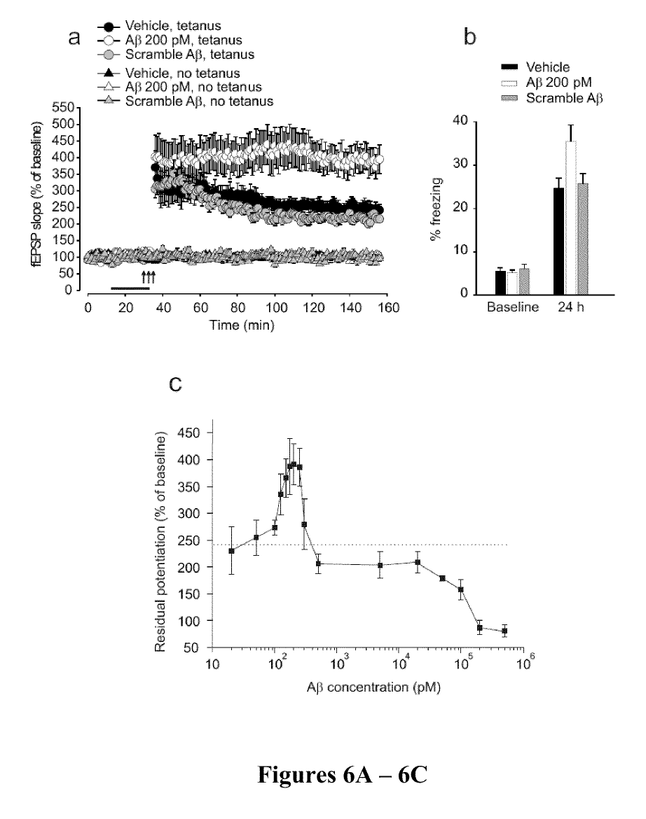

The invention provides that Aβ at physiological concentrations, acts to enhance LTP in vitro and fear memory in vivo. The invention provides preparations containing oligomeric human Aβ₄₂, which, when used at concentrations similar to those found in vivo in the brain, enhance LTP and fear memory retention. In addition, sequestration of endogenous Aβ by antibody binding reduces LTP and fear memory retention. These findings suggest that endogenously produced neuronal Aβ may play a critical role during memory formation. However, the broadly held dogma is that Aβ is likely to be neurotoxic, to reduce synaptic function, and to decrease the ability to consolidate memory [72, 73]. For example, it has been shown that oligomeric Aβ₄₂ can inhibit LTP in higher concentrations [1-6], a finding reproduced here (see FIG. 6C). Indeed, it is generally thought that increased levels of Aβ lead to synaptic dysfunction in β-amyloid depositing transgenic mouse models (for a review see [11]), and that the resulting behavioral deficits can be rapidly diminished following anti-Aβ immunotherapy [74-76].

Exogenous Aβ, at concentrations thought to be physiological in the brain, and endogenous Aβ at its normal concentration can play a positive role in LTP and both contextual fear memory and IA memory. Given that Aβ exists in different forms that have been shown to act differently on synaptic transmission (i.e. monomers do not affect plasticity, whereas oligomers and especially dodecamers markedly impair LTP [77] and memory [78]), the invention provides what form(s) of Aβ can mediate this effect.

The role of endogenous Aβ in LTP and memory and determining the form of Aβ-Aβ₄₂ vs. Aβ₄₀—that mediates these effects was assessed. The outcome of these in vitro studies provided the details of the design and execution (e.g. Aβ concentration, timing of Aβ injection into the hippocampi relative to training) of the in vivo studies in mice and rats, where memory retention was determined using the contextual fear learning and IA paradigm, respectively.

These findings show that LTP is enhanced by the addition of a preparation containing oligomeric Aβ₄₂ at low concentrations (200 pM; see Example 6a), and further studies show that the injection of this same Aβ₄₂ preparation enhances memory retention in mice and rats (see FIGS. 6b and 15c). Dose response curves can be used to assess the effect on LTP of oligomeric Aβ₄₂ from concentrations of 20 pM to 500 nM ([1-6], see also FIG. 6c) to show that Aβ at high concentrations is inhibitory while determining the optimal lower concentration for LTP enhancement by oligomeric Aβ₄₂. Experiments can be used to determine whether the addition of oligomeric Aβ₄₂ is effective during the induction or also after the tetanus during the maintenance of LTP because mechanisms leading to LTP are different than those underlying its maintenance. In parallel, both an in vivo mouse- and rat-model can be used to determine whether the injections of oligomeric Aβ₄₂ are effective when administered when the memory is partially or more fully consolidated. Toward this end, one can test the effect of Aβ₄₂ injected before and after training and test memory retention at 2 and 48 hours after training. In behavioral experiments where memory enhancement is found, one can determine whether the effect is long-lasting by re-testing the animals 1 and 3 weeks after training. If at 3 week the enhancement is still found, the animals can be re-tested at 8 weeks after training. This time point should also reveal whether Aβ₄₂ treatment also promotes long-term retention of fear memory event (reduces “forgetting”).

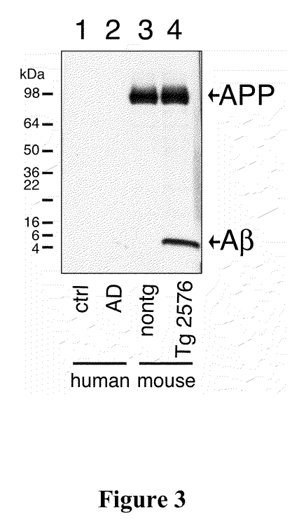

Additionally, acute, antibody-mediated depletion of the endogenously produced Aβ has been found to dramatically interfere with LTP in vitro and both contextual fear learning and IA memory formation in vivo (see Examples 5 and 15). The antibody used in these studies is the anti-rodent Aβ monoclonal antibody m3.2 (m3.2 mAb) which was generated by vaccinating APP null mice with rodent Aβ-derived peptides. This antibody recognizes an epitope within residues 11-15 of the rodent Aβ sequence (FIG. 3), a region that contains two aa differences when compared to the human sequence. This antibody binds synthetic and endogenous, brain-derived rodent Aβ (both Aβ₄₀ and Aβ₄₂) with high affinity by ELISA and by immunoprecipitation, and completely fails to recognize human Aβ (FIG. 3). The epitope for m3.2 is also contained within APP holoprotein and the alpha-cleavage APP fragment known as sAPPa. This is demonstrated in the Western blot shown in FIG. 3, where m3.2 fails to bind to any APP metabolite in human control or AD brain, but recognizes the endogenous murine APP in a wt mouse (lane 3). In addition, in an aged APP transgenic mouse with robust β-amyloid deposition, which allows for detection of the abundant deposited Aβ by simple Western blot analysis, m3.2 detects co-deposited murine Aβ (lane 4). This binds with high affinity rodent Aβ, and interacts with native-conformation rodent Aβ. More than 10 laboratories have independently confirmed the usefulness of m3.2 to bind and detect rodent Aβ. No cytotoxicity has been seen when primary cultures of rat neurons have been grown in the presence of 5 mg/ml m3.2 containing media for 48 hrs, nor has ip injection of 0.5 mg m3.2 weekly over an 8 week period shown any toxic effects in wt or human APP transgenic mice. In addition to m3.2, monoclonal antibodies are available that specifically react with the C-terminus of Aβ₄₀ or Aβ₄₂. While these antibodies have high specificity for Aβ₄₀ vs. Aβ₄₂, they, like all C-terminal Aβ-directed antibodies, do not differentiate between the human and rodent peptides.

The invention provides that oligomeric Aβ₄₂ at concentrations that are similar to those expected in vivo enhances LTP and memory. By sequestering the endogenously generated Aβ using antibodies, experiments directly demonstrate that an endogenous Aβ form plays an important role in mediating LTP and memory. The m3.2 antibody is a monoclonal antibody that binds rodent Aβ with high affinity under multiple conditions (non-denaturing, denaturing, monomeric, oligomeric/fibril). Taking advantage of m3.2's absolute specificity for the rodent peptide, human Aβ rescue experiments performed in the presence of m3.2 should show that the important APP metabolite mediating this effect is Aβ. The LTP response to exogenous Aβ in APP null mice can also be investigated. A correlation can be made of LTP findings with the contextual fear learning and IA memory paradigm in vivo. This in vitro and in vivo approach can supply compelling information on Aβ-form specificity, mechanism, and biological relevance. Aβ endogenously generated Aβ at its appropriate concentration in the appropriate environment in the non-diseased brain plays a critical role in memory.

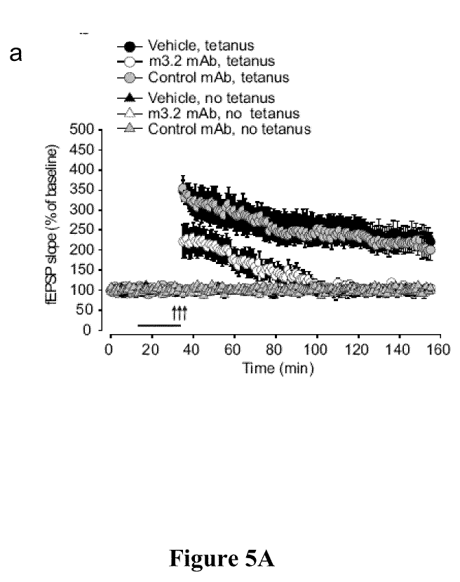

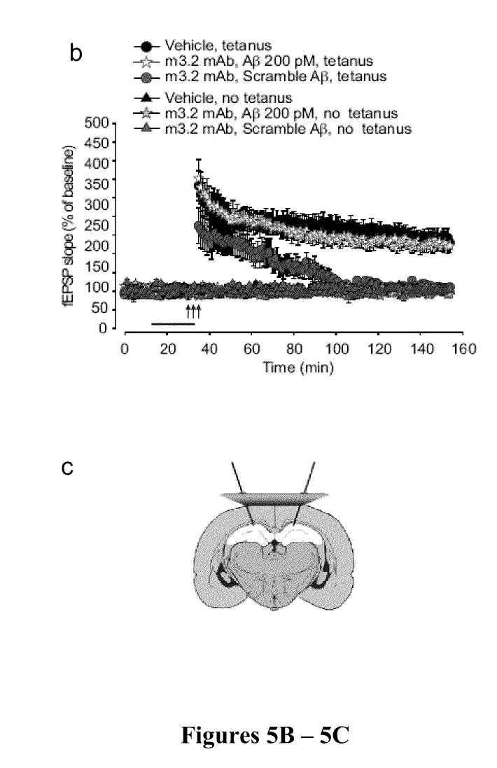

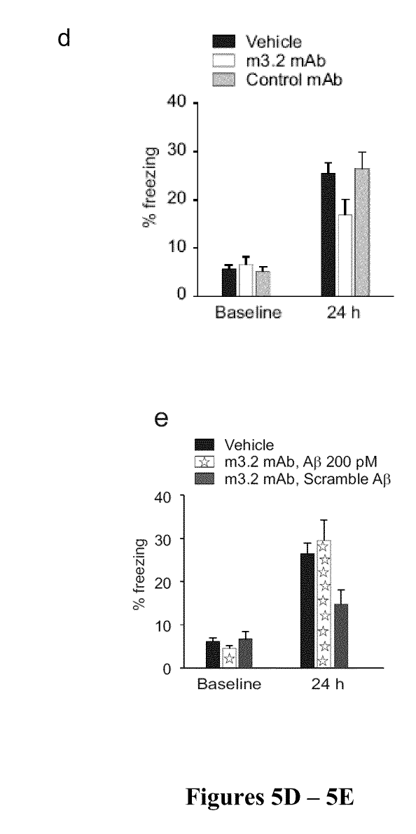

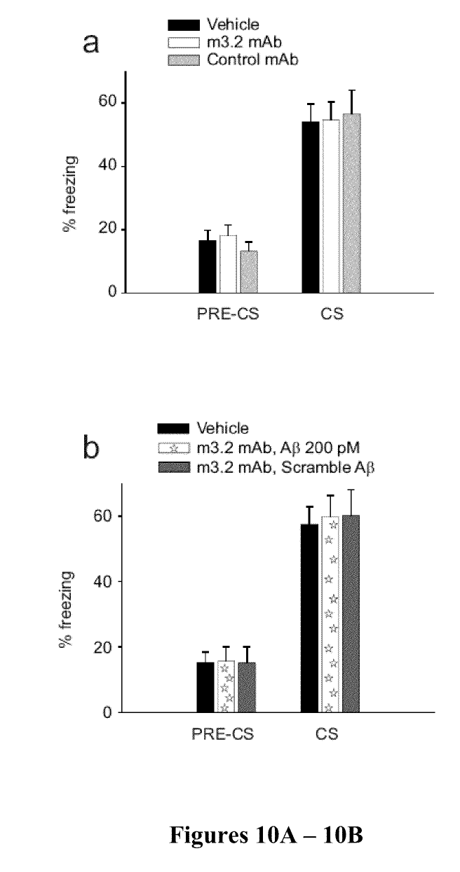

In both slices and behavioral systems, the ability of m3.2 antibody to specifically interfere with Aβ can be assessed by performing rescue experiments in which exogenous oligomeric human Aβ₄₂, which is not recognized by the m3.2 antibody, is added concurrent with the anti-rodent-Aβ m3.2 antibody, confirming that the effect is specifically mediated by Aβ. These experiments demonstrate that endogenous Aβ at its physiological concentration impacts LTP and memory retention. Control experiments have made use of the control mAb (as in FIG. 5B, FIG. 5D and FIG. 10A).

One can determine whether the effects are specific for Aβ₄₂, the molecule that nucleates and drives production of amyloid fibrils [79], or mediated also by Aβ₄₀, which represents the majority of secreted Aβ peptides. Oligomeric Aβ₄₀ can be prepared and tested in both systems. If an effect of Aβ₄₀ is found, its specific role can be confirmed by using anti-Aβ₄₀-specific antibodies and related controls. Likewise, effects unique to Aβ₄₂ can be confirmed using anti-Aβ₄₂-specific antibodies.

Experiments can be done to determine whether an observed effect is mediated by monomeric Aβ and/or oligomeric Aβ. Methods can be done to see whether Aβ dodecamers specifically are capable of mediating the LTP enhancement seen with oligomeric Aβ₄₂. Experiments can be performed examining the effect of a preparation containing exclusively either monomers or oligomers on LTP, with in vivo studies done in follow-up to positive LTP findings.

Methods for obtaining detailed electrophysiological recordings on brain slices are known in the art [4, 5, 67, 80-86]. Methods for assessing memory formation in vivo, including the use of direct hippocampal injections and analysis of underlying molecular mechanisms are known in the art [87-89].

Amyloid Beta Precursor Protein

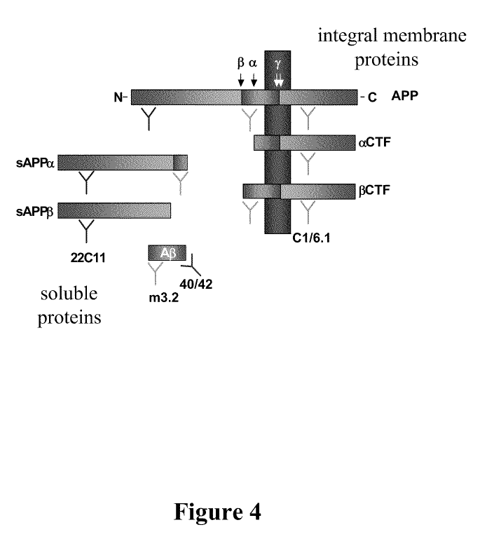

Aβ is a proteolytic fragment of the larger amyloid-β precursor protein (APP) [18-22], a type-1 transmembrane protein that resembles a cell surface receptor [19] and contains a large extracellular domain, a hydrophobic transmembrane domain and a short intracellular domain ([23, 24]; see FIG. 4). APP undergoes proteolytic processing by three proteases, the so called α- β- and γ-secretase [24, 25]. APP is initially cleaved by β- or β-secretases, generating large, soluble, secreted fragments (sAPPα and sAPPβ) and membrane-associated carboxy-terminal fragments (CTFs; see FIG. 4). These CTFs are substrates for the γ-secretase, and sequential β-secretase cleavage followed by γ-secretase cleavage in the transmembrane domain leads to the production of the various length A-beta peptides (e.g. Aβ₄₀ and Aβ₄₂).

The normal function of APP remains poorly understood, although the sAPP fragments may have neurotrophic properties [26-30] and the intracellular CTF generated after γ- and the related β-cleavage may regulate gene transcription [31-33]. A-beta itself has been suggested to have some function(s), although in general it is largely considered by the field as a “garbage” fragment generated during the production of other, biologically important APP fragments [16]. A-beta has received the most attention as a key pathological protein in AD [16], which has been clearly underscored by the understanding of the genetics of the rare, early-onset forms of familial AD (FAD) caused by mutation in three genes—APP, presenilin (PS) 1 and PS2 (see [34] for review and details). A unifying feature of these mutations is that they increase the production of A-beta or increase the generation of a particularly pathogenic or aggregation prone A-beta species (such as Aβ₄₂).

APP expression increases in the process of differentiation and growth of neurons [35], suggesting a role in nervous system development, which has been confirmed by behavioral and plasticity studies [36]. APP might also be involved in several memory mechanisms [37] including LTP [38] (also see Doyle et al, (1990) Neurosci Letters 115:97-102; Huber et al., (1993) Brain Res 60:348-352; Mileusnic et al., (2000) Eur J Neurosci 12:4487-4495). Indeed, loss of APP function by knock-out has demonstrated an impairment of both LTP and memory in mice [39-41]. A knock-out approach, however, does not permit one to distinguish between the effects of different APP fragments. Although sharing sequence homology with other members of the APP protein family (the APP-like proteins 1 and 2 in mammals [42-44]), only APP contains an A-beta-like domain, suggesting that A-beta may have a unique physiological function in vivo. Neuronal activity appears to positively modulate the secretion of A-beta peptides [45, 46], which has been shown to lead to synaptic depression in systems overexpressing FAD-mutant APP [46]. The loss of presenilin function, the enzymatic subunit of the multicomponent γ-secretase protein complex [47], also impairs LTP and memory. This is accompanied by both pre- and post-synaptic changes such as a reduction in paired pulse facilitation correlating with a decreased number of total and docked vesicles, together with a reduction both in NR1 and NR2A subunits of the NMDA receptor and in activation of CaMKII, and a reduction in nuclear c-Fos levels [48, 49]. However, because of the diverse substrates of the γ-secretase in addition to APP (an important example is Notch [47]), it remains to be determined through what mechanism(s) A-beta or otherwise—loss of presenilin function causes these synaptic effects. Nevertheless, these findings make a strong case for a role for A-beta in synaptic plasticity and memory. The invention provides the disclosure of a positive role for physiological, not pathological, levels of A-beta in the regulation of LTP and memory.

The invention also provides methods to determine whether APP metabolism is altered in vitro following tetanus and in vivo following contextual fear learning and IA, promoting the transient generation of increased amounts of Aβ. Methods for assessing APP metabolites and Aβ levels in the rodent CNS are known in the art [58-70].

The invention provides that APP metabolism is altered in vitro following tetanus and in vivo following contextual fear learning and IA, promoting the transient generation of increased amounts of A-beta. Metabolism of endogenous APP is altered during the processes that lead to synaptic plasticity and memory formation. Levels of APP and APP metabolites, including A-beta, are altered following tetanus in hippocampal slices and both contextual fear learning and IA training in vivo.

The m3.2 antibody experiments show that Aβ endogenously produced by hippocampal slices and in the rat brain plays a critical role in LTP and both contextual fear learning and IA memory formation. A recent study [45] has shown that in vivo stimulation of the perforant pathway leads to increased Aβ levels in the brain interstitial fluid of Tg2576 human APP transgenic mice, as measured by in vivo microdialysis. These findings are in agreement with an earlier study in which hippocampal slices from human APP transgenic mice were shown to release more Aβ following stimulation [46]. Importantly, these prior studies have used transgenic mice that express familial, early onset-AD Swedish-mutant APP, which has alterations in its β-secretase processing and the amount of Aβ generated from a given pool of APP molecules. Endogenous Aβ levels and the levels of various APP metabolites—all generated from the wild-type, normally expressed and processed rodent APP are measured. Experiments can be designed to determine whether the levels of endogenous Aβ increase after stimulation, whether alterations in APP processing by the secretases or other changes in APP metabolism are responsible for an increase in Aβ levels, or, as has been proposed in the APP transgenic systems [45], whether release of Aβ from intracellular pools may account for an increase in Aβ following stimulation.

Experiments can be designed to determine whether the metabolism of endogenous APP is altered during the processes that lead to LTP and memory formation, examining both of the experimental systems (tetanus in hippocampal slices and both contextual fear learning and IA training in vivo). The levels of APP and the known APP metabolites (sAPPα; sAPPβ; αCTF; βCTF; Aβ) can be characterized from homogenates prepared from the CA1 region of tetanized mouse hippocampal slices and from hippocampal homogenates prepared from dissected brains of mice that were trained for contextual fear learning and rats that were trained with the IA test.

α7-Nicotinic Acetylcholine Receptors (nAchRs)

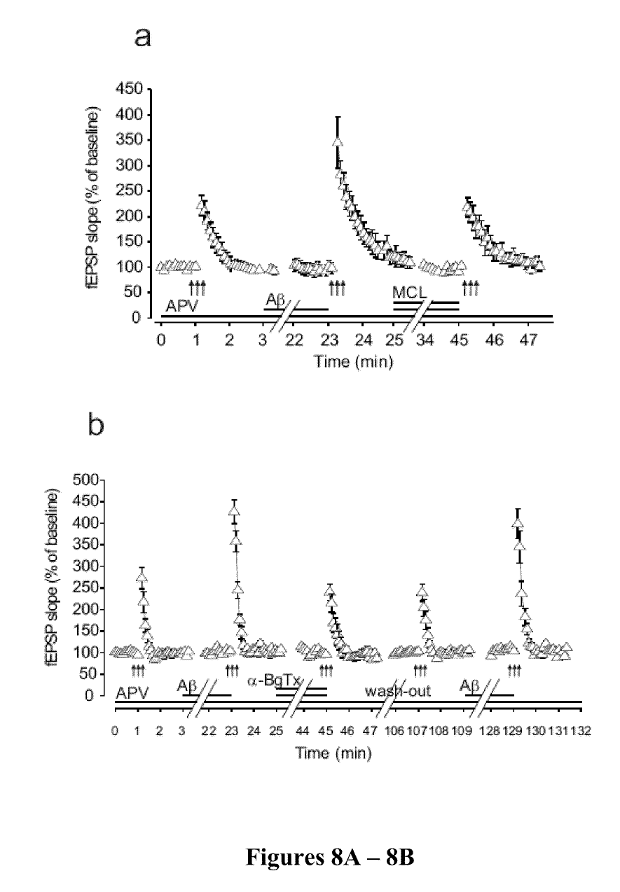

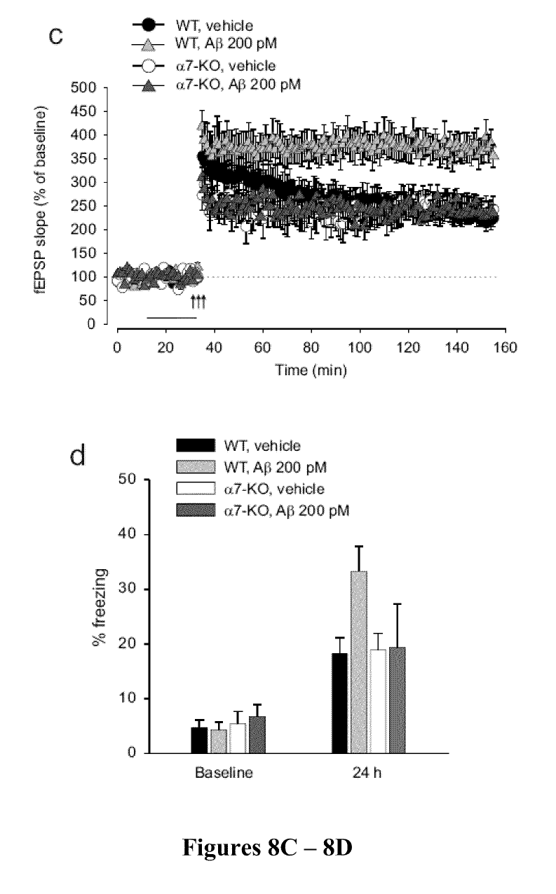

The invention provides for methods to enhance α7-nicotinic acetylcholine receptors (nAchRs) responses by a low concentration of oligomeric Aβ₄₂. Neuronal α7-nicotinic acetylcholine receptors, a multigene family of ligand gated ion channels that are involved in diverse brain functions including synaptic plasticity and memory [117, 118], have been shown to bind to Aβ₄₂ in the low picomole range [119]. Aβ₄₂, in turn, activates α7/(β2-nAchRs at presynaptic nerve endings of synaptosomes when administered in the low picomole range (whereas it blocks nAchR activation at somatic sites in the more classically used nanomole range [120, 121]). The experiments shown on FIGS. 8A, 8B, 8C, and 8D show that the enhancing effect by Aβ₄₂ on post-tetanic potentiation, LTP and contexual fear learning is due to α7-nAchRs, as they are blocked either by the non specific nAchR blocker MCL, or the specific α7-nAchR blocker, α-bungarotoxin (α-BgTx), or knocking-down the α7-nAchR in null mice.

It is a finding of the invention that sequestration of endogenous Aβ by antibody binding reduces LTP and contextual fear memory. In contrast to the known impairment in LTP and memory due to high levels of Aβ, the invention provides that a preparation containing oligomeric human Aβ₄₂, when used at concentrations that similar to those found in vivo in the brain, enhances LTP and associative memory. In addition, physiologically relevant concentrations of Aβ enhance PTP—a type of short-term plasticity that is believed to be an indication of presynaptic function and reflects a period of enhanced transmitter release during the tetanus—but not affect post-synaptic AMPA receptors. Activation of pre-synaptic α7-nAchRs is likely involved in the enhancement of synaptic plasticity and memory as loss of α7-nAchR function blocks their increase, whereas chronic depletion of APP in KO mice is associated with a dramatic enhancement of LTP by physiologically relevant levels of Aβ₄₂. Based upon these findings positive and negative effects of Aβ on synaptic function and memory may represent a continuum: the negative effects of Aβ at higher concentrations in the diseased brain are in part due to the overwhelming of a more subtle role Aβ plays at the synapse in the non-diseased brain. See FIG. 6.

Methods of Treatment

The invention provides methods for treating a subject by administering an amyloid beta peptide.

In one embodiment, the subject may be suffering from a memory disorder. In another embodiment, the subject may be normal and be undergoing treatment to enhance normal memory. In another embodiment, the subject may have Alzheimer's Disease, head trauma, or an attention deficit disorder. The invention also provides that the memory disorder comprises or is associated with Alzheimer's disease, Parkinson's disease, Pick's disease, a Lewy body disease, amyotrophic lateral sclerosis (ALS), Familiar Alzheimer's Disease, Parkinson-ALS-dementia complex of Guam and other island areas, Cerebellar degenerations, Huntington's disease, Creutzfeld-Jakob disease, Down syndrome, multiple system atrophy, neuronal degeneration with brain iron accumulation type I (Hallervorden-Spatz disease) and other rare genetic diseases (Kufs', Wilson's, late-onset metachromatic leukodystrophy, adrenoleukodystrophy), pure autonomic failure, REM sleep behavior disorder, progressive supranuclear palsy (PSP), corticobasal degeneration, progressive myoclonic epilepsy, mild cognitive impairment (MCI), cerebral amyloid angiopathy (CAA), vascular dementias (multi-infarct dementia, strategic single infarct dementia, small-vessel disease with dementia, hypoperfusion dementia, hemorrhagic dementia, Binswanger disease), endocrine and metabolic disorders with dementia, malnutrition dementia, Wernicke-Korsakoff Syndrome, alcohol dementia, Vitamin B12 and folate deficiency, Toxic dementia (Metallic dementia e.g. lead, mercury, arsenic, manganese; Organic poisons e.g., solvents, some insecticides), Traumatic dementia, Infectious dementia (Acquired immune deficiency syndrome, Opportunistic infections, subacute spongiforn encephalopathy, Progressive multifocal leukoencephalopathy, Post-encephalitic dementia, Behcet's syndrome, Herpes encephalitis, Bacterial meningitis or encephalitis, Parasitic encephalitis, Neurosyphilis), space-occupying lesions (chronic or acute subdural hemtoma, primary or metastatic brain tumor), auto-immune disorders (multiple slerosis, lupus erythematosus, vasculitis), amnestic disorder not otherwise specified (NOS), amnesia related to schizophrenia, mood disorders, anxiety, substance abuse; Dementia Alzheimer's type with delirium, delusions, depressed mood; delusional misidentification syndrome, learning disabilities, Attention Deficit Hyperactivity Disorder (ADHD) mixed with Alzheimer's disease, a neurodegenerative disease characterized by abnormal amyloid deposition, mild cognitive deficits, aging or any combination thereof.

The low dose of amyloid beta peptide can be any amount which will provide a local concentration of from about 50 pM to about 300 pM in the hippocampal tissue of the subject. This concentration will result from optimization of routes of delivery and from optimization of formulation of the A-beta drug, or A-beta derivative.

In connection with the method of this invention, a therapeutically effective amount of the inhibitor may include dosages, which take into account the size and weight of the subject, the age of the subject, the severity of the symptoms, the method of delivery of the agent and the history of the symptoms in the subject. In one embodiment, the dose of the amyloid beta 42 peptide, or a therapeutic variant thereof, is that which will produce a local concentration (in the relevant brain tissue) of from about 125 pM to about 500 pM, or from about 130 pM to about 480 pM, or from about 140 pM to about 475 pM, or from about 150 pM to about 450 pM, or from about 160 pM to about 440 pM, or from about 170 pM to about 430 pM, or from about 180 pM to about 420 pM, or from about 190 pM to about 410 pM, or from about 200 pM to about 400 pM, or from about 210 pM to about 350 pM, or from about 200 pM to about 300 pM, or from about 200 pM to about 225 pM, or from about 200 pM to about 250 pM, or from about 200 pM to about 275 pM.

The invention provides for the maintenance of a low concentration of Aβ in the brain of a subject. In one embodiment, the maintained concentration of Aβ is about 200 pM. In another embodiment, the maintained concentration of Aβ is about a physiological concentration of Aβ. Maintenance can include, for example, increasing a concentration of Aβ or decreasing a concentration of Aβ to achieve a physiological concentration. Increasing Aβ concentration may be achieved, for example, with compounds that modify APP processing in physiologically beneficial ways, compounds that change Aβ levels through secretases, or compounds that inhibit Aβ degrading enzymes.

The invention provides for delivery of an Aβ peptide (such as Aβ₄₂) to the brain of a subject via direct administration or otherwise. Direct administration to the brain can be via various methods, which would be known to one of skill in the art. For example, direct administration could be via a pump device placed through the skull of the subject. Another method of direct administration is via direct injection into the brain. The invention also provides for delivery of the peptide by use of a carrier molecule or agent. A carrier can be a molecule or agent that will target the brain. For example, a targeting molecule can be a molecule that enables the peptide or peptidomimetic of the invention to cross the blood-brain-barrier. The peptide can be delivered to the brain via delivery of a nucleic acid which encodes the peptide, wherein the nucleic acid is expressed in the brain and therefore the peptide delivered into the brain. In a further embodiment, the nucleic acid can comprise a sequence, which encodes the peptide, and can further comprise a neural specific nucleic acid that specifically targets the peptide to a neural cell. In one aspect of the invention, the promoter is a constitutive promoter. In another aspect, the promoter is a neural cell-specific promoter.

The administration of the amyloid beta peptides of the invention may be effected by intralesional, intraperitoneal, subcutaneous, intramuscular or intravenous injection; by infusion; or may involve liposome-mediated delivery; or sublingual, topical, nasal, oral, anal, ocular or otic delivery.

In the practice of the method, administration of a compound may comprise daily, weekly, monthly or hourly administration, the precise frequency being subject to various variables such as age and condition of the subject, amount to be administered, half-life of the agent in the subject, area of the subject to which administration is desired and the like. In one embodiment, administration of Aβ is in accord with a schedule that maintains a concentration of Aβ in the brain of a subject, wherein the maintained concentration is at or near the physiological concentration of Aβ.

In one embodiment, the concentration of Aβ in a subject is monitored so as to maintain a certain concentration of Aβ in the subject.

The invention provides that Aβ itself is a critical factor in synaptic plasticity and memory in the normal brain. Indeed, the use of drugs that mimic Aβ structure, or that are targeted to the receptor(s) on which Aβ acts under normal physiological conditions, or even of Aβ itself or Aβ derivatives may serve to enhance memory at appropriate concentrations.

Another consequence of the fact that Aβ itself is a critical factor in synaptic plasticity and memory in the normal brain, is that a reduction of Aβ levels below normal physiological levels can produce a loss of synaptic function and memory, such that drugs aiming at reducing Aβ levels might have negative effects if Aβ levels are reduced below a certain level.

The Examples below provide in vitro and in vivo approaches that supply information on Aβ-form specificity, mechanism, and biological. The experiments described in this application supply strong experimental evidence that Aβ endogenously generated Aβ at its appropriate concentration in the appropriate environment in the non-diseased brain is a critical factor in synaptic plasticity and memory within the normal brain.

As stated above, the following references are hereby incorporated by reference in their entireties.

The following examples illustrate the present invention, and are set forth to aid in the understanding of the invention, and should not be construed to limit in any way the scope of the invention as defined in the claims which follow thereafter.

### EXAMPLES

### Example 1

### Detection of Endogenous Aβ in Rat Brain Regions

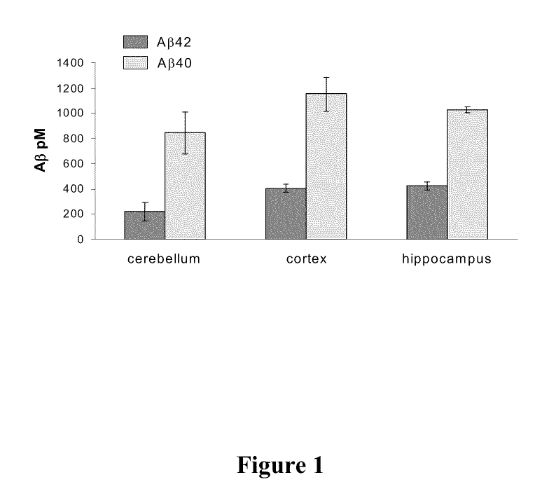

The invention provides antibody-based assays that detect the endogenously expressed APP in rodent brain, along with the endogenous rodent APP metabolites: soluble APP, a-cleaved (sAPPα); soluble APP, β-cleaved (sAPPβ); the C-terminal membrane associated APP fragment, α-cleaved (αCTF); C-terminal fragment, β-cleaved (βCTF); total Aβ; Aβ₄₀; Aβ₄₂ (see FIG. 4, which is a cartoon illustrating these APP metabolic fragments). Western blot, immunoprecipitation, and sandwich ELISA assays [58-70] are examples of methods capable of detecting subtle changes in Aβ levels in the rodent brain. Similar assays were applied to examine APP metabolism and Aβ levels following tetanus in hippocampal slices and IA training in vivo. Quantitation of the levels of the endogenous rat Aβ from extracts prepared from isolated cerebellum, cortex, and hippocampus is shown in FIG. 1. These brains regions were dissected, flash frozen, homogenized and extracted by diethylamine (DEA) [65, 66]—prior to sandwich ELISA to quantitate Aβ₄₀ and Aβ₄₂. These levels of Aβ₄₂ (200-400 pM) and Aβ₄₀ (1000 pM) detected in rat brain following DEA extraction, which is an alkaline, denaturing extraction that recovers soluble and oligomeric/fibrilar Aβ, are consistent with the levels previously reported in mouse brain [59, 61, 63, 64, 66, 68, 71] and are consistent with a report using in vivo microdialysis (estimated to be approximately 700 pM Aβ40 in the mouse brain; [14]). These findings support the use of exogenous Aβ₄₂ in a range of 200 pM as approximating the endogenous physiological concentration. One can examine the effect of Aβ₄₂ and Aβ₄₀ on LTP, contextual fear learning and IA across a range of concentrations, including those concentrations that are physiological (˜200 pM Aβ₄₂, ˜1000 pM Aβ₄₀). Importantly, one can deplete the endogenous Aβ using the m3.2 anti-Aβ antibody in experiments that make no presuppositions about the physiological concentration of Aβ.

### Example 2

### APP Metabolism

Hippocampal slices can be prepared with four CA1 regions isolated and pooled from slices for each experimental condition. Tetanus is applied, and the CA1 region harvested after various time points to compare APP metabolite levels to control non-tetanized slices. Initial time course studies (e.g., 2, 5, 10, 15, 60, 120 min after tetanus) for the most readily detectable cell-membrane-associated APP metabolites (e.g. APP, CTFs) can be done to establish the condition that shows the maximal change in APP metabolism. Once such conditions are established, additional experiments to examine the levels of each of the APP metabolites can be done. This more detailed analysis can use 9-10 slices pooled from three individual mice per condition to yield sufficient tissue for analysis.

APP metabolism in the rat brain can be examined following IA. A comparison of a limited number of naïve rats and trained rats (n=3 each group), at various time points following training (5, 15, 30 min) can be done. The choice of initial time points can be guided by findings with oligomeric Aβ₄₂ and m3.2 antibody injection in the hippocampus (FIGS. 5 and 15), which show that the modulation of memory by Aβ occurs close to training. Additionally, in vivo findings indicate that the half-life of Aβ in the rodent brain is short, less than 20 min. In initial studies, levels of all of the APP metabolites outlined above can be assessed, as is routinely done in the laboratory from rodent brain [58, 59, 61, 63-66, 68, 103-105]. Once the time course is established, additional experiments can be done with more rats (final n=5 each condition) and can include, in addition to naïve and trained cohorts, a control group that is exposed to the environmental context without shock and a control group that receives foot-shock without context association. Immediately following sacrifice, brain tissue can be dissected and the two hippocampi from each rat pooled and flash frozen for later homogenization and analysis [65]. Cortex and cerebellum can be used as control regions.

Given that endogenous Aβ can play a crucial role in LTP and memory formation, one can expect that these events will be associated with an increase in Aβ production by the neuron. Given that Aβ is generated through β- and gamma-secretase proteolytic steps, one would further anticipate that an increase in Aβ production would be accompanied by changes in the levels of other APP fragments generated by these secretases. Indeed, an analysis of APP metabolites can indicate whether increased levels of Aβ are the result of increased Aβ production or simply the release of Aβ from pre-existing intracellular, vesicular pools. An analysis of the various APP metabolites following tetanus may suggest whether an increase in the production of Aβ, is the result of a generalized increase in the processing of APP (which would be reflected in acutely lower levels of APP and increased levels of both the alpha- and beta-cleavage products), a recruitment of BACE activity and increased β-cleavage of APP (increased β-cleaved fragments, decreased a-cleaved fragments), or a recruitment of γ-secretase activity (increased Aβ, no change in sAPPα or sAPPβ, decreased CTF levels). One could further examine the mechanisms—changes in protein trafficking, localization, protease function—that could lead to an increase in Aβ production by the neuron in response to stimulation. The comprehensive analysis of APP metabolites will allow one to develop a complete picture of the impact of the stimuli on APP metabolism and Aβ generation, suggesting that not only can Aβ modulate memory, but that Aβ levels are regulated during the events that lead to memory formation.

### Example 3

### α7-nAchR Measurements

Given that activation of α7-nAchRs presynaptic receptors is known to elevate Ca²⁺ levels [122] and facilitate glutamate release [123], an increase in Aβ₄₂ during tetanus may enhance plasticity and memory. Studies can be done to examine whether the preparations used following Aβ treatment show enhanced hippocampal Ca²⁺ levels in synaptomes [120, 121]. Then, to determine whether α7-nAchRs are responsible for this effect, the α7 blocker, α-bungorotoxin, can be used (see FIG. 8). A report showing an effect of low picomoles of Aβ₄₂ on α7-nAchRs [120], also demonstrates that the stimulating effect of Aβ₄₂ was also dependent upon α7-nAchR channels in the low picomole range but not at nanomole range. Thus, Aβ₄₂ at physiologically relevant concentrations may also interfere with α7-nAchR channels.

### Example 4

### Aβ₄₂ is Likely to Act During the Induction Phase of LTP

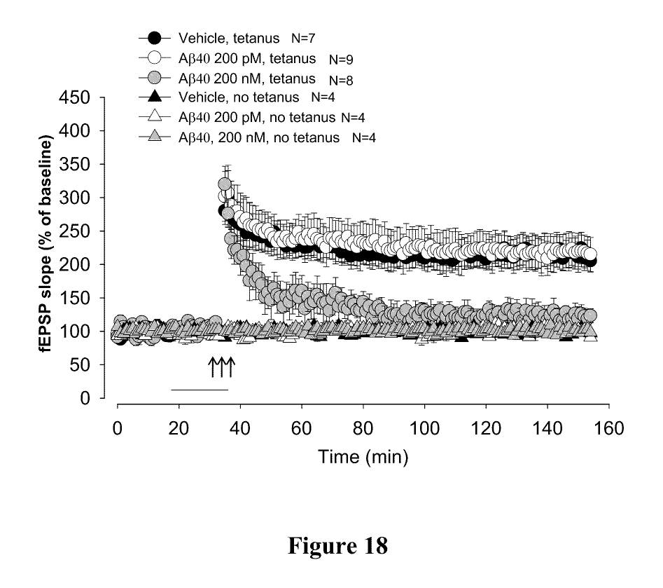

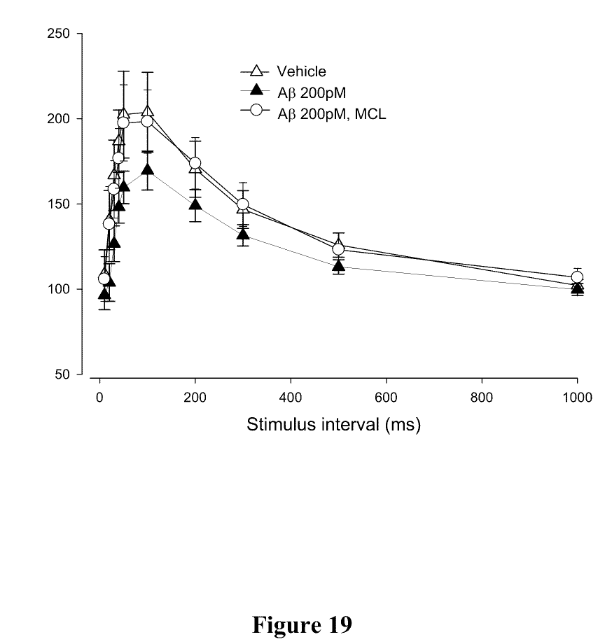

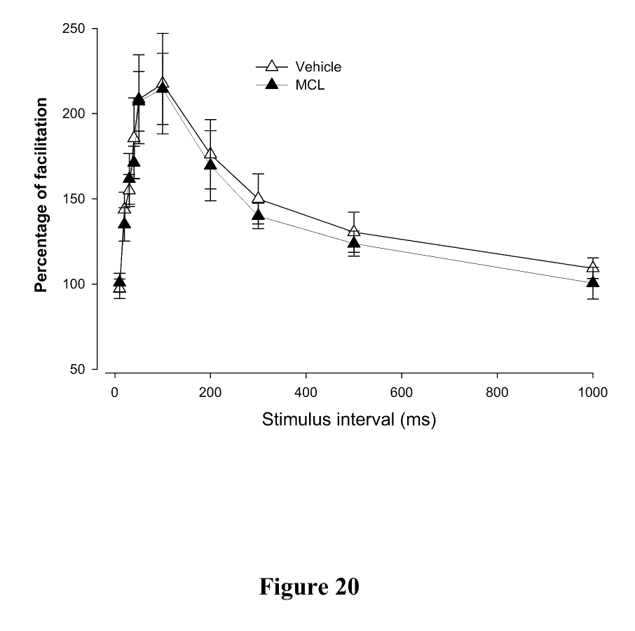

Given that Aβ exists in different forms that have been shown to act differently on synaptic transmission (i.e. monomers do not affect plasticity, whereas oligomers and especially dodecamers markedly impair LTP [77] and memory [78]), experiments were designed to determine what form(s) of Aβ can mediate the effect enhancing synaptic plasticity and memory. In addition to Aβ₄₂, Aβ₄₀ is normally produced in the brain. When Aβ₄₀ was applied at a concentration of 200 nM (i.e. a dose equal to that capable of producing synaptic dysfunction), the peptide was capable of markedly reducing LTP at high concentrations (200 nM), while at physiological concentration (200 pM) it did not affect the amount of potentiation (200 nM: 123.44±25.45%, n=8, F(1, 13)=10.82, p<0.01; 200 pM: 215.02±25.45%, F(1, 15)=0.23, n=9, p>0.05; vs 205.28±16.69% at 120 min after tetanus, n=7; FIG. 18). Neither 200 nM nor 200 pM Aβ₄₀ concentration affected baseline synaptic transmission (200 nM: 98.82±1.82%, n=4, F(1, 6)=0.05, p>0.05; 200 pM: 91.25±4.05%, n=4, F(1, 6)=1.07, p>0.05; vs 95.51±1.42% at 120 min after tetanus, n=4; FIG. 18). Thus, these results suggest that oligomeric Aβ₄₀ is not capable of enhancing synaptic transmission at physiologically relevant concentrations.

### Example 5

### Human Aβ₄₂ Rescues the Loss of LTP and Fear Memory Induced by a Rodent Aβ Antibody

To acutely knock-down the function of the whole APP, the m3.2 mAb was used. This antibody recognizes an epitope within residues 11-15 of the rodent Aβ sequence, a region that contains two amino acid differences when compared to the human sequence. This antibody binds synthetic and endogenous, brain-derived rodent Aβ with high affinity by ELISA (FIG. 3) and by immunoprecipitation, and completely fails to recognize human Aβ (FIG. 3). The epitope for m3.2 is also contained within the APP holoprotein and the α-cleavage APP fragment known as sAPPα. This is demonstrated in the Western blot shown in FIG. 3, where m3.2 fails to bind to any APP metabolite in human control or AD brain, but recognizes the endogenous murine APP in a wild-type mouse (lane 3). In addition, in an aged APP transgenic mouse with robust Aβ deposition, which allows for detection of the abundant deposited Aβ by simple Western blot analysis, m3.2 mAb detects co-deposited murine Aβ (lane 4).

To test whether m3.2 mAb impair synaptic plasticity, hippocampal slices were perfused with m3.2 mAbs diluted in the bath solution at a concentration of 2 μg/ml for 20 min prior to tetanizing the slices. The treatment dramatically suppressed LTP (105.28±5.81 vs 220.77±21.88% of baseline slope in vehicle-treated slices at 120 min after tetanus, n=10/8; two-way ANOVA F(1,16)=67.35, p<0.001 FIG. 5a). As a control, we perfused hippocampal slices for 20 min prior to tetanization with a IgG1a mAb (2 μg/ml) that does not bind to any rodent proteins (our anti-human-specific, intracellular epitope PS1 mAb, NT1, termed control mAb (Mathews et al., 2000)). This treatment did not affect LTP (200.70±26.64; n=6; F(1,12)=0.016, p=0.903 compared to vehicle-treated tetanized slices; FIG. 5a). Non-tetanized slices treated with m3.2 mAb alone or with control mAb alone showed no change in basal synaptic transmission (m3.2 mAb: 99.36±3.99%, n=5; control mAb: 105.37±7.47%, n=4; vehicle: 103.68±2.40%, n=5; F(1,8)=1.09, p=0.325, and F(1,7)=0.29, p=0.606, both compared to non-tetanized slices perfused with vehicle; FIG. 5a).

Given that m3.2 mAb recognizes not only Aβ but also APP and sAPPα, rescue experiments were performed to confirm that its effect is specifically mediated by Aβ. Physiologically relevant concentrations of Aβ₄₂ in mouse brain were determined by measuring the endogenous mouse peptide levels from isolated cerebellum, cortex, and hippocampus (FIG. 1). Levels of Aβ₄₂ ranging from 200 to 400 pM (Mastrangelo et al., 2005; Pawlik et al, 2004; Phinney et al, 2003; Rozmahel et al., 2002a; Rozmahel et al., 2002b; Schmidt et al., 2005b; Yao et al., 2004). Addition of exogenous oligomeric human Aβ₄₂ (200 pM) which is not recognized by the antibody, concurrent with the anti-rodent-Aβ m3.2 mAb, rescued the LTP defect (221.79±18.28 vs 229.89±18.48% of vehicle-treated slices; n=8/8; two-way ANOVA F(1,16)=0.316, p=0.583; FIG. 5b). Antibody m3.2 paired with Aβ₄₂ without tetanization did not affect the baseline (99.76±2.29%, n=4; F(1,6)=0.45, p=0.523 compared to non-tetanized vehicle-treated slices, n=5; FIG. 5b). As a control for nonspecific effects of the peptide, a peptide consisting of scrambled Aβ₄₂ sequence (Malin et al., 2001) failed to rescue the damage of LTP by m3.2 mAb (110.77±5.10%, n=7; F(1,9)=4.28, p=0.069 compared to non-tetanized vehicle-treated slices, n=5; FIG. 5b). Taken together, these findings show that endogenously produced Aβ plays a critical role in LTP induction.

Given that LTP is thought to represent an electrophysiological correlate of learning and memory, the same strategy as for the electrophysiological experiments was used to assess the effects of Aβ on contextual fear memory (Phillips and LeDoux, 1992), a form of associative learning for which hippocampus is indispensable. Cannulas were implanted bilaterally into the mouse dorsal hippocampi (FIG. 5c). After 6-8 days, animals were infused with the m3.2 mAb (1 μg in 1 μl), control mAb (1 μg in 1 μl) or vehicle, respectively, and after 15 min trained to associate neutral stimuli with an aversive one. No difference was detected in the freezing behaviour among the three groups of mice during the training phase of the fear conditioning (FIG. 5d). However, when fear learning was assessed twenty-four hours later by measuring freezing behavior—the absence of all movement except for that necessitated by breathing—in response to representation of the context, m3.2 mAb-treated mice showed a decrease in the freezing behaviour (16.83±3.25% of freezing vs 25.44±2.23% of freezing in vehicle-treated mice, n=18/16, t(32)=2.20, p=0.034; FIG. 5d). By contrast, control mAbs did not affect freezing (26.47±3.28%, n=13, t(27)=0.194, p=0.848 compared to vehicle-treated mice; FIG. 5d). In interleaved experiments, 200 pM Aβ₄₂—but not scrambled Aβ₄₂—concurrent with m3.2 mAbs re-established normal freezing (29.51±4.78% vs 26.44±2.50% in vehicle-injected mice; n=13/16, t(27)=0.770, p=0.448; 14.84±3.31 s in scramble Aβ₄₂+m3.2 mAb injected mice; n=10, t(24)=2.77, p=0.011 compared to vehicle-injected mice; FIG. 5e). Moreover, when cued fear learning—a type of learning that depends upon amygdala function (Phillips and LeDoux, 1992)—was assessed twenty-four hours after contextual learning by measuring freezing in response to representation of the auditory cue within a completely different context, no difference was detected between vehicle-infused mice and both m3.2 mAb infused mice (t(32)=0.097, p=0.924; FIG. 10a), and m3.2 mAb+Aβ₄₂ infused mice (F(1,27)=0.59, p=0.557; FIG. 10b), indicating that behavioral changes produced by the antibodies and their rescue by Aβ₄₂ were due to a selective hippocampus-dependent effect on associative learning. Finally, when mice were trained and, immediately after, bilaterally infused with m3.2 mAbs, no effect was detected on contextual learning 24 hours later (25.56±6.50, n=5, t(8)=0.07, p=0.943 compared to vehicle-infused mice). Taken together, these findings indicate that similar to LTP endogenously produced Aβ plays a critical role in associative memory.

### Example 6

### Physiologically Relevant Levels of Aβ₄₂ Enhance LTP and Fear Memory

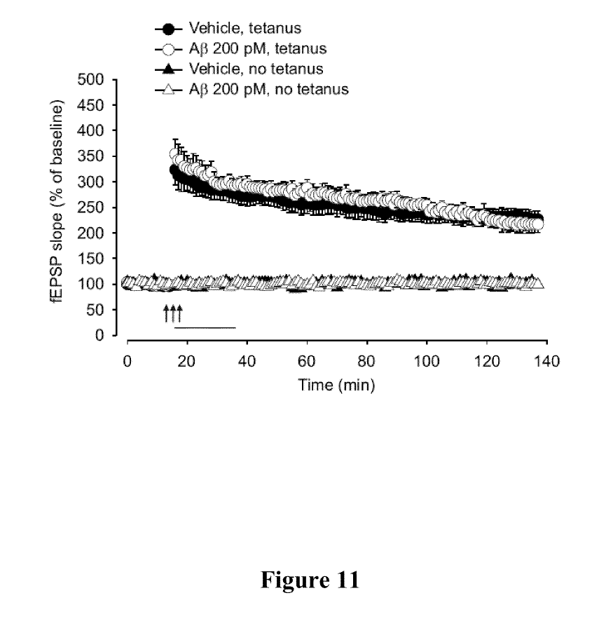

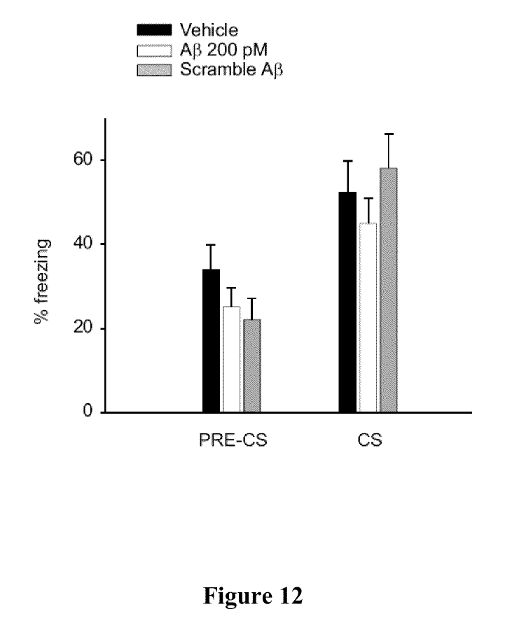

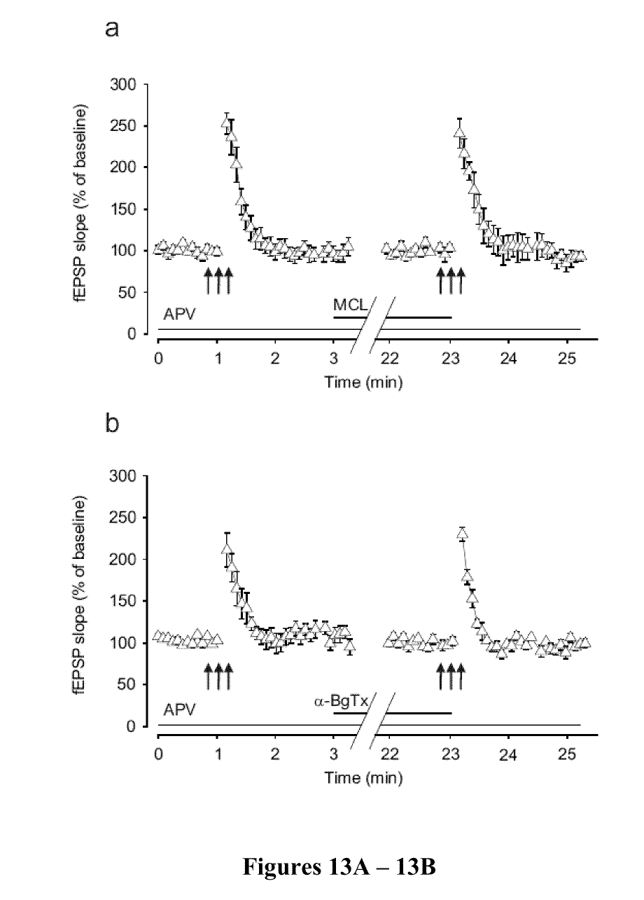

A series of experiments were conducted to determine whether Aβ per se has an effect on synaptic plasticity and associative memory in an environment in which there is no depletion of its levels. 200 pM Aβ₄₂ was applied for 20 min before tetanization of the Schaeffer collateral-CA1 connection in slices. An increase of the amounts of potentiation was found (349.95±43.29 vs 241.79±17.25% in vehicle-treated slices; n=12/10; two-way ANOVA F(1,20)=7.20, p=0.014; FIG. 6a). Aβ₄₂ alone did not affect basal transmission (103.09±3.16%, n=6; F(1, 12)=0.05, p=0.814 compared to vehicle-treated slices, n=8; FIG. 6a), nor did application of Aβ₄₂ for 20 min immediately after the tetanus (217.27±16.64 vs 226.53±16.70% in vehicle-treated slices, n=5/5; two-way ANOVA F(1,8)=0.34, p=0.573; FIG. 11). In control experiments, scrambled Aβ₄₂ did not affect LTP (215.01±9.62%, n=6), nor baseline transmission (96.19±8.39%, n=4; FIG. 6a). Consistent with these results, infusion of 200 pM Aβ₄₂ into hippocampi enhanced freezing (35.52±16.61% vs 24.73±2.27% in vehicle-injected mice, n=19/17, t(34)=2.36, p=0.024; FIG. 6b). By contrast, scrambled Aβ₄₂ did not affect freezing (25.78±2.23%, n=15, t(30)=0.33, p=0.743 compared to vehicle-injected mice; FIG. 6b). Moreover, cued fear learning was similar between Aβ₄₂-infused mice and vehicle-infused mice (434)=0.269, p=0.79; FIG. 12), indicating that behavioral changes produced by Aβ₄₂ were due to a selective hippocampus-dependent enhancement in associative learning. Taken together, these experiments show that Aβ₄₂ at low concentrations, similar to the physiologic concentrations of the peptide, causes a long lasting increase in synaptic strength and enhances associative memory.

The increase in LTP by Aβ₄₂ was unexpected, as the peptide is known to reduce potentiation (Cullen et al., 1997; Itoh et al., 1999; Klyubin et al., 2005; Vitolo et al., 2002; Walsh et al., 2002). Therefore, to check whether the preparation of Aβ₄₂ was still capable of impairing LTP at the usually utilized higher concentrations, hippocampal slices were perfused with different concentrations of Aβ₄₂. The preparation of oligomeric Aβ₄₂ reduces LTP when used at higher concentrations than those present in the non-diseased brain (FIG. 6c). The amounts of potentiation in slices that were perfused with 200 nM Aβ₄₂ were equal to 87.27±13.12% (n=7) and significantly lower than in vehicle-treated slices (234.54±19.82%, n=12, two-way ANOVA F(1,17)=27.56, p<0.0001). Moreover, the concentration/response curve was “bell-shaped” with a peak around 200 pM, corresponding to the concentration that enhanced LTP and memory in the experiments shown above.

### Example 7

### Physiologically Relevant Levels of Aβ₄₂ Enhance PTP

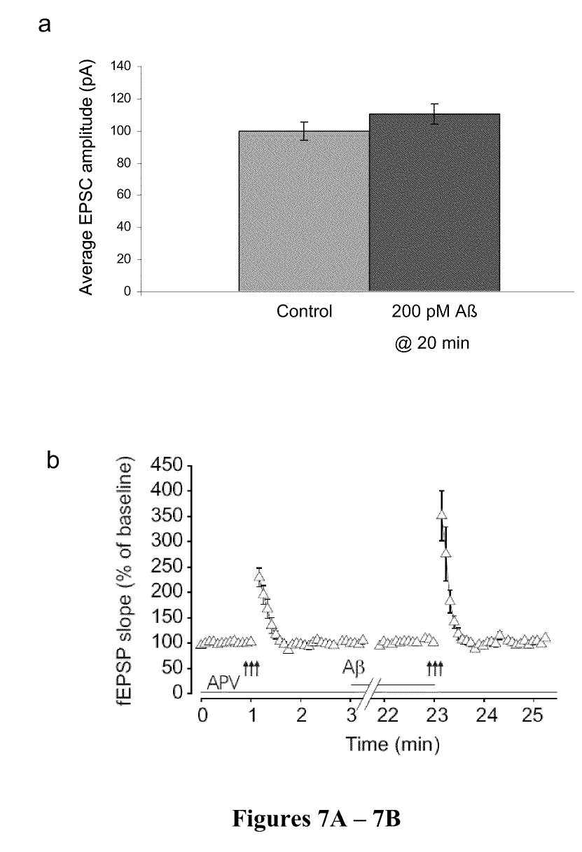

Experiments were designed to identify the cause of the Aβ-induced enhancement in LTP and fear memory. Given that AMPA receptors play a key role in LTP (Bliss and Collingridge, 1993), experiments were designed to determine whether evoked AMPA receptor currents were modified by perfusion of the slices with 200 pM Aβ₄₂ for 20 min using patch clamp technique. AMPA receptor currents were evoked by stimulating the Shaffer collateral pathway with a concentric bipolar electrode placed in stratum radiatum and recording with a patch electrode from the cell body of CA1 piramidal neurons. Aβ did not affect the amplitude (FIG. 7a) of AMPA receptor mediated EPSCs at 20 min after perfusion with Aβ, indicating that the enhanced LTP was not caused by post-synaptic changes in AMPA receptor currents. An alternative mechanism for the long-lasting enhancement in synaptic strength might be an increase of transmitter release during the tetanus. To test this possibility, post-tetanic potentiation (PTP), a type of short-term plasticity that is believed to be an indication of presynaptic function and reflects a period of enhanced transmitter release during the tetanus was assessed (Zucker and Regehr, 2002). PTP was induced in the presence of the NMDA antagonist D-APV (50 pM) to block LTP inductive mechanisms. Perfusion of the slices with 200 pM Aβ₄₂ for 20 min produced an increase in PTP (153% of PTP before Aβ perfusion at the first time point after the tetanus; n=11, t(20)=2.18, p=0.041; FIG. 7b), suggesting that Aβ enhances transmitter release during the tetanus.

### Example 8

### α7-nAchRs are Necessary for the Enhancement of Synaptic Plasticity and Fear Memory Induced by Physiologically Relevant Levels of Aβ₄₂

Several lines of evidence suggest that the Aβ-induced increase in PTP is due to a modulation of α7-nAchR activity during the tetanus. Activation of α7-containing nicotinic acetylcholine receptors (α7-AChRs), a multigene family of ligand gated ion channels that are involved in diverse brain functions including synaptic plasticity and memory (Jones et al., 1999; Levin and Simon, 1998), enhances transmitter release in several brain structures including the hippocampus (Gray et al., 1996), the spinal cord dorsal horn (Genzen and McGehee, 2003), and in hippocampal neurons in culture (Radcliffe and Dani, 1998), as well as co-cultures of olfactory bulb neurons and amygdala neurons (Girod et al., 2000). Nicotinic activity at pyramidal neurons boosts LTP induction (Ji et al., 2001). Studies examining binding of Aβ₄₂ to AChRs indicate that Aβ has a picomolar affinity for α7-AChRs (Wang et al., 2000) or might regulate nAChR function through membrane lipids (Small et al., 2007). Aβ₄₂, in turn, activates α7-AchRs at presynaptic nerve endings of synaptosomes when administered in the low picomole range (whereas it blocks nAchR activation at somatic sites in the more classically used nanomole range (Dougherty et al., 2003; Nichols, 2006)). The “bell-shaped” concentration-response curve around the picomolar concentration shown on FIG. 6C resembles that of nicotinic agonists.

To determine if the Aβ-induced increase in PTP is involved in the modulation of α7-nAchR activity during the tetanus, hippocampal slices were perfused with the non-selective nAchR blocker mecamylamine (MCL) (3 μM for 20 min). The drug blocked the Aβ-induced PTP increase (99% of PTP before Aβ perfusion at the first time point after the tetanus; n=11, t(20)=0.11, p=0.91) in slices that had previously shown an enhancement of PTP when perfused with Aβ alone (156% of PTP before Aβ perfusion; 420)=2.15, p=0.044, FIG. 8a). Moreover, the selective α7-nAchR blocker, α-bungarotoxin (α-BgTx) (0.1 μM for 20 min) also blocked the Aβ-induced PTP increase (88% of PTP before Aβ perfusion at the first time point after the tetanus; n=10, t(18)=1.02, p=0.317) in slices that had previously shown an enhancement of PTP when perfused with Aβ alone (156% of PTP before Aβ perfusion; t(18)=6.78, p<0.001, FIG. 8b). The slices were still capable of displaying the Aβ-induced PTP enhancement if perfused again with Aβ alone after wash-out of both α-BgTx and Aβ (147% of PTP before Aβ perfusion at the first time point after the tetanus; t(18)=2.93, p=0.009; FIG. 8b). Furthermore, both the PTP increase by Aβ alone and its block by α-BgTx were still present in the presence of the GABA receptor antagonists bicuculline (10 μM) and phaclophen (0.1 mM) (93% of PTP before Aβ perfusion at the first time point after the tetanus in slices perfused with the GABA antagonsists plus Aβ; n=7, t(12)=5.70, p<0.001), suggesting that α7-nAchRs located in inhibitory interneurons are not involved in the Aβ-induced enhancement of transmitter release occurring during tetanic stimulation.

To eliminate the function of α7-nAchRs by a non-pharmacological nAchR-specific approach, α7-nAchR knockout (KO) mice were used. When hippocampal slices from these animals were perfused with 200 pM Aβ₄₂ for 20 min prior to inducing LTP at the Schaffer collateral-CA1 connection with the tetanus, the peptide failed to enhance LTP (242.90±27.83 vs 234.28±18.17% in vehicle-treated α7-KO slices; n=9/8; two-way ANOVA F(1,15)=0.001, p=0.975; FIG. 8c). By contrast, Aβ₄₂ was still capable of enhancing LTP in slices from wild-type littermates (355.43±24.11 vs 223.70±16.84% in vehicle-treated WT slices; n=7/7; two-way ANOVA F(1,12)=9.04, p=0.11; FIG. 8c). Aβ alone without tetanus did not affect baseline transmission both in α7-KO- and WT slices (105.95±3.30 and 102.39±3.49%, respectively; n=4/4). Similarly, hippocampal infusion of 200 pM Aβ₄₂ failed to enhance contextual fear memory in α7-KO mice (19.4±7.88% vs 19±2.98% in vehicle-injected α7-KO mice, n=3/3, t(4)=0.04, p=0.964; FIG. 8d), whereas it still increased memory in WT littermates (33.31±4.39% vs 18.25±2.94% in vehicle-injected WT mice, n=8/6, t(12)=2.63, p=0.022; FIG. 8d). This confirms that α7-nAchRs are involved in the enhancing effect of Aβ on synaptic plasticity and fear memory.

### Example 9

### Chronic Depletion of APP in KO Mice is Associated with a Dramatic Enhancement of LTP Following Exposure to Physiologically Relevant Levels of Aβ₄₂

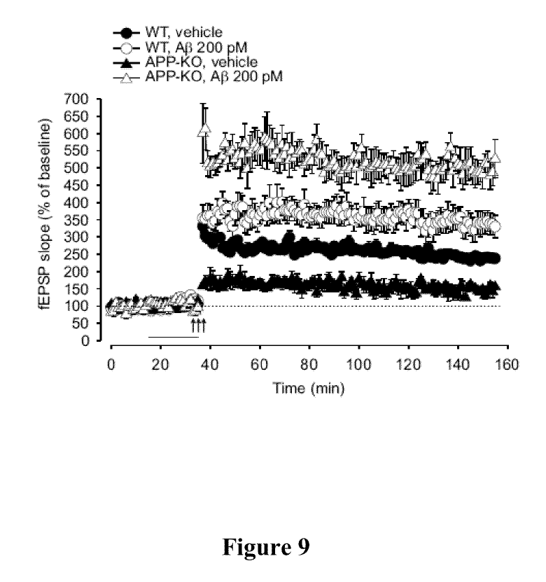

Consistent with the observation that m3.2 mAbs impair LTP and fear memory, APP-KO mice have an impairment of both LTP and memory (Dawson et al., 1999; Phinney et al., 1999; Seabrook et al., 1999). Therefore, studies were conducted to test whether Aβ is capable of rescuing the deficit of LTP in these mice. 200 pM Aβ₄₂ for 20 min prior to the tetanus produced a much bigger increase than expected (527.43±50.89 vs 157.65±11.06% in vehicle-treated APP-KO slices; n=7/6; two-way ANOVA F(1,11)=119.30, p<0.0001; FIG. 9). By contrast, the increase in LTP by Aβ in WT littermates was of the same amount as in previous experiments with slices from WT littermates (337.33±29.44 vs 227.78±5.35% in vehicle-treated WT slices; n=6/6; two-way ANOVA F(1,10)=9.70, p=0.011; FIG. 9A). Aβ alone without tetanus did not affect baseline transmission both in APP-KO- and WT-slices (95.45±8.46 and 97.99±4.10%, respectively; n=4/4).

### Example 10

### Physiological Function of Aβ in Fear Based Memory

Long-Evans rats were implanted with cannulas that bilaterally targeted the hippocampi and one week was provided for recovery from surgery. In a series of experiments, the animals were infused with the m3.2 mAb (1 μg in 1 μl) or control mAb (1 μg in 1 μl) and after 15 min trained on inhibitory avoidance. As shown with contextual fear conditioning, assessment of memory retention 24 hours after training showed a dramatic memory disruption in m3.2 mAb animals (163.1±60.1 s vs 409.5±37.7 s in control mAb, n=12/11; p<0.001; one-way ANOVA followed by Neuman-Keuls post-hoc test; FIG. 15a). By contrast, if rats were bilaterally injected immediately after training with m3.2 Ab or control mAb and memory retention was tested after 24 hours, no effect was found and both groups of rats showed similar memory retention (m3.2 Ab: 306.2±77.9 s; control mAb: 270.1±47.2 s; n=8 for both, p>0.05). 200 pM human Aβ₄₂—but not scrambled Aβ₄₂—re-established normal memory retention in animals that were also injected with m3.2 mAbs (FIG. 15b). In another series of experiments, fear conditioning in rats injected with 200 pM Aβ₄₂ confirmed the memory enhancement observed in peptide-injected mice (see FIG. 15c). Memory retention was significantly enhanced in rats that received Aβ₄₂ (FIG. 15c), whereas 200 pM scrambled Aβ₄₂ did not change memory retention (FIG. 15c). Taken together these results are consistent with data obtained with contextual fear learning, indicating that endogenously produced Aβ plays a critical role in memory and low concentrations of Aβ₄₂ at the appropriate physiological concentration enhance associative memory in vivo.

### Example 11

### Methods

Rats. Long Evans adult male rats (Harlan) weighing 200-250 g at the beginning of procedures were used in all experiments. Rats were individually housed and maintained on a 12 h on/12 h off light/dark cycle and underwent behavioral procedures during the light cycle.

Surgical procedure in rats. Rats were anesthetized with ketamine (60 mg/Kg; i.p) and xylazine (7.5 mg/Kg; i.p) and implanted with cannulas (22 gauge; Plastics One, VA) positioned 1.5 mm above the hippocampus using the following coordinates (Paxinos, 1998): 4.0 mm posterior to Bregma; 2.6 mm lateral from midline; 2.0 mm ventral. Rats recovered for 7 days after surgery before undergoing any experimental procedures (Garcia-Osta et al., 2006).

Injections in rats. Aβ peptides and control peptides were diluted in PBS to reach the appropriate concentration immediately before use. They were infused bilaterally through the infusion cannulas at 0.4 μl/min using a pump, and a total volume of 1 μl was infused into each side. Additional controls were injected with the same volume of the vehicle solution (PBS). The injection cannulas were left in place for at least 1 min to allow the solution to completely outflow from the cannula tip.

Behavioral procedures in rats. The inhibitory avoidance training procedure was performed as previously described (Garcia-Osta et al., 2006; Taubenfeld et al., 2001). In this task, the animals form an association between a context and a shock, and consequently avoid the context at subsequent exposures. The task is known to require an intact hippocampus and amygdala (Ambrogi Lorenzini et al., 1999; Izquierdo and Medina, 1997; McGaugh, 2002). One advantage for using this task is that a strong and long-lasting memory occurs following a single training trial, which allows an accurate temporal analysis of the molecular events and requirements that take place during the different phases of learning and memory. This task has been widely utilized to identify and characterize the molecular bases of memory consolidation and reconsolidation [94-99]. The inhibitory avoidance apparatus (Med. Assoc.) consisted of a rectangular-shaped Perspex box, divided into a safe compartment and a shock compartment. The safe compartment was white and illuminated; the shock compartment was black and dark. Foot shocks were delivered to the grid floor of the dark chamber via a constant current scrambler circuit. The apparatus was located in a sound-attenuated, non-illuminated room. Rats were handled once a day for 3 days before behavioral experiments. During training sessions, each rat was placed in the safe compartment with its head facing away from the door. After 10 s, the door separating the compartments was automatically opened allowing the rat to access the shock chamber. Latency to enter the shock chamber was taken as a measure of fear acquisition. The door closed 1 s after the rat entered the shock chamber, and a brief foot shock (0.9 mA for 2 s) was administered. The rat was then removed from the apparatus and returned to its home cage. Inhibitory avoidance memory was tested at the desired time points after training Testing consisted of placing the rat back in the safe compartment and measuring the latency to enter the shock chamber. If rats did not enter the dark compartment, testing was terminated at 540 s. Foot shock was not administered during the retention test.

Histology in rats. At the end of the behavioral experiments, rats were anesthetized with Ketamine (60 mg/kg; i.p.) and Xylazine (7.5 mg/Kg; i.p.) and perfused intracardially with 10% PBS-buffered formalin. The brains were removed, post-fixed overnight in a 30% sucrose/formalin solution and then cryo-protected in 30% sucrose/saline. To verify the location of the cannula implants, 40 μm coronal sections were cut through the targeted area, stained with cresyl violet and examined under a light microscope.

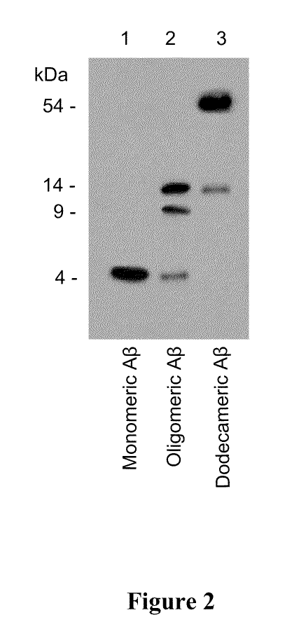

Aβ₄₂ preparation. Oligomeric Aβ₄₂ was prepared as previously described (Puzzo et al, 2005). Briefly, the lyophilized peptide (American Peptide) was suspended in 100% 1,1,1,3,3,3-hexafluoro-2-propanol (HFIP; Sigma, St. Louis, Mo.) to 1 mM. HFIP was allowed to evaporate and the resulting clear peptide film stored at 20° C. Twenty-four hours before use, the film was added to dimethylsulfoxide (DMSO; Sigma) and sonicated for 10 min. Aβ₄₂-DMSO was diluted into the bath solution, vortexed for 30 sec, and incubated at 4° C. for 24 hrs. This synthetic Aβ has been extensively characterized both biochemically and electrophysiologically, demonstrating similar biological effects at low nanomolar concentrations as naturally secreted oligomers of Aβ (Puzzo et al., 2005; Trommer et al., 2005; Walsh et al., 2002; Wang et al., 2004). In addition, its normal sequence—but not the scrambled sequence—blocks LTP rapidly, robustly and consistently (Puzzo et al., 2005), indicating that the electrophysiological action of Aβ can be readily assayed before major compensatory effects, inflammatory response, neuritic degeneration or apoptosis have occurred. Scramble Aβ₄₂ was purchased from AnaSpec Inc. (San Jose, Calif.), and prepared as for Aβ₄₂. Oligomeric Aβ₄₀ is obtained following the same procedure but increasing the incubation time to 8 weeks at 4° C. [100]. Oligomers can be isolated by size-exclusion chromatography (SEC) [101] as described below. Dodecameric Aβ₄₂ can be obtained according to a slight modification of the method proposed by Barghorn et al. [77] derived from the original Stine's preparative protocol for the oligomeric Aβ [100]. Briefly, an aliquot of monomeric Aβ₄₂ is diluted with SDS 0.2% in sterile PBS and stored at 4° C. overnight. Then, the dodecameric Aβ₄₂ solution is also purified by SEC to isolate the dodecameric fraction. The quality of these Aβ preparations is routinely controlled by Western blot analysis. Briefly, Aβ samples are resolved by Tris-Tricine PAGE [102] under non-denaturing/non-reducing conditions, and then transferred on nitrocellulose membrane. Subsequent Western blotting is carried out after membrane incubation with the anti-human Aβ monoclonal antibody 6E10 (Signet Lab). The immunostaining is revealed by horseradish peroxidase chemioluminescence (see FIG. 2).

SEC. To physically separate different Aβ forms, Aβ samples prepared as above can be run on two Superdex 75 prep grade 20×500 mm columns (˜100 ml volume) arranged in series and eluted with 50 mM ammonium acetate pH 8.5. Fractions are collected via fast protein liquid chromatograph (FPLC), lyophilized and stored at −20° C. Individual SEC column fractions can be resuspended and diluted at the final concentration for electrophysiological and behavioral experiments. Alternatively, the fractions can be resuspended in 1× Tricine sample buffer, and half-fractions run on SDS-PAGE as described above.

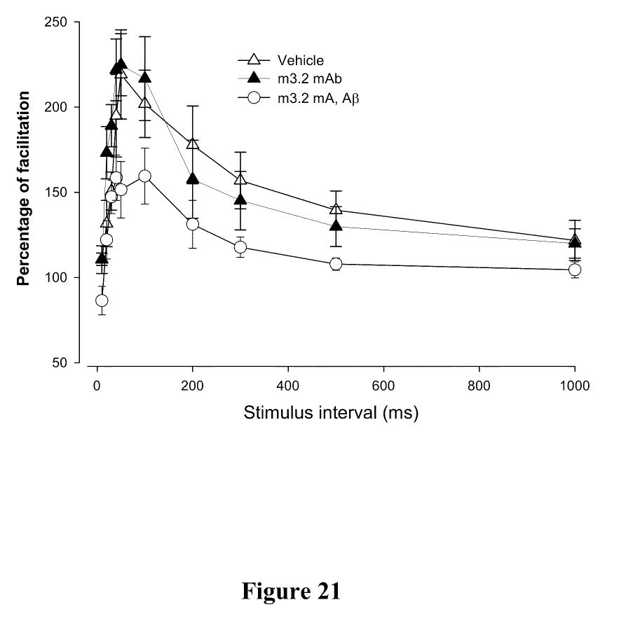

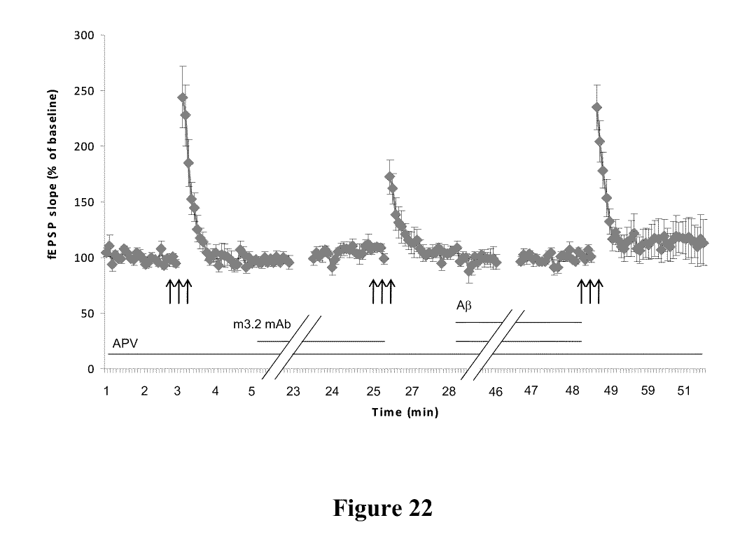

Anti-Aβ antibodies. The anti-rodent Aβ monoclonal antibody m3.2 (m3.2 mAb) was generated by vaccinating APP null mice with rodent Aβ-derived peptides. No cytotoxicity has been seen when primarily cultures of rat neurons have been grown in the presence of 5 μg/ml m3.2 containing media for 48 hrs, nor has ip injection of 0.5 mg m3.2 mAb weekly over an 8 week period shown any toxic effects in wild-type or Tg2576 APP transgenic mice. The working concentration of 2 μg/ml used in the electrophysiology experiments is based upon the concentration of m3.2 Ab typically employed in the laboratory for immunoprecipitation, and the amount injected into animals (1 μg/hippocampus) was calculated from this and has been empirically determined to be effective (see FIGS. 5, 15, 21 and 22).

Aβ measurements. Brain regions were dissected, flash frozen, homogenized and extracted by diethylamine (DEA)—as previously described (Schmidt et al., 2005a; Schmidt et al., 2005b)—prior to sandwich ELISA to quantitate Aβ₄₂. Levels of Aβ₄₂ were detected following DEA extraction, which is an alkaline, denaturing extraction that recovers soluble and insoluble Aβ.

Biochemical detection of endogenous rodent APP metabolites from slice and brain preparations. Levels of APP metabolites, including Aβ, will be determined as is routine in our laboratory from cell lysates and brain homogenates (as described [65, 103, 105, 107]), using antibodies (e.g. m3.2, C1/6.1) and additional monoclonal antibodies (anti-Aβ40, anti-Aβ42) (22C11; see FIG. 3). APP holoprotein levels will be determined by Western blot of total protein homogenates using antibody C1/6.1 (FIG. 3). Levels of CTFs, both a- and b-cleaved, will also be determined by Western blot analysis using this antibody. The soluble APP fragments (sAPP) are detected after membrane proteins are removed by centrifugation [65] using 22C11, or for sAPPα, monoclonal antibody m3.2. Levels of sAPPβ are determined by immunoprecipitation with m3.2 to remove sAPPα and then Western blot of the remaining material with 22C11 to detect sAPPβ. Endogenous rodent Aβ levels (Aβ40 and Aβ42) will be determined by the sandwich ELISAs [65, 66].

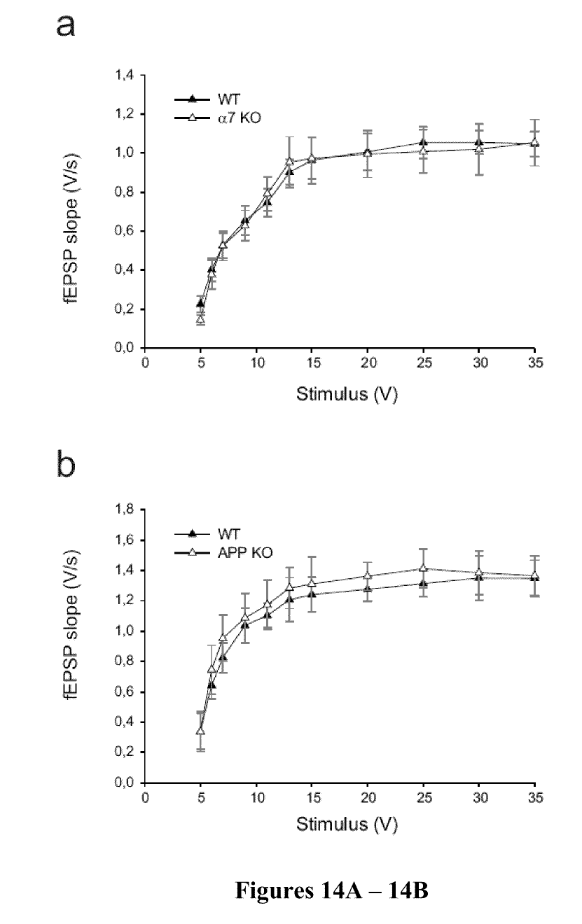

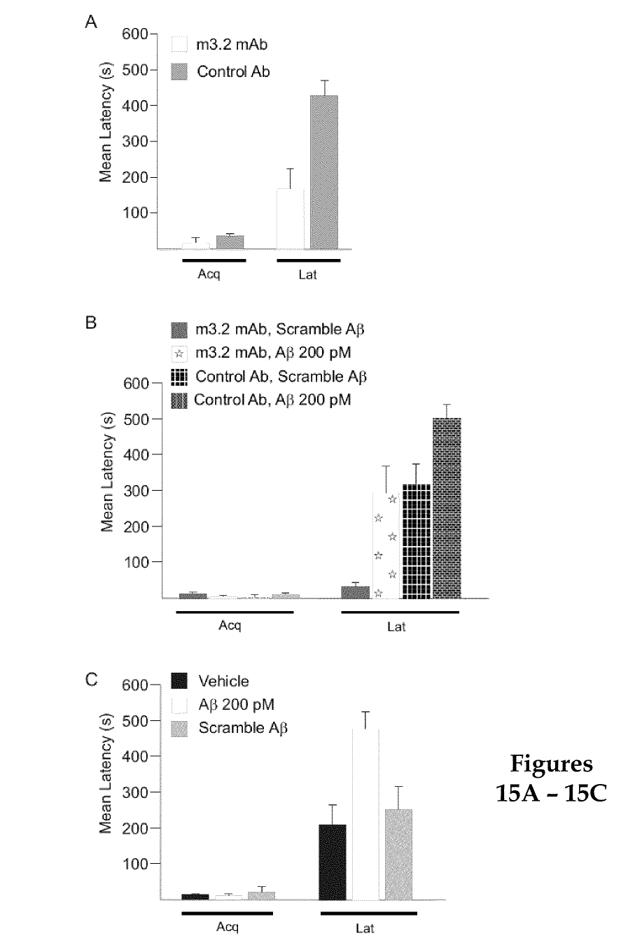

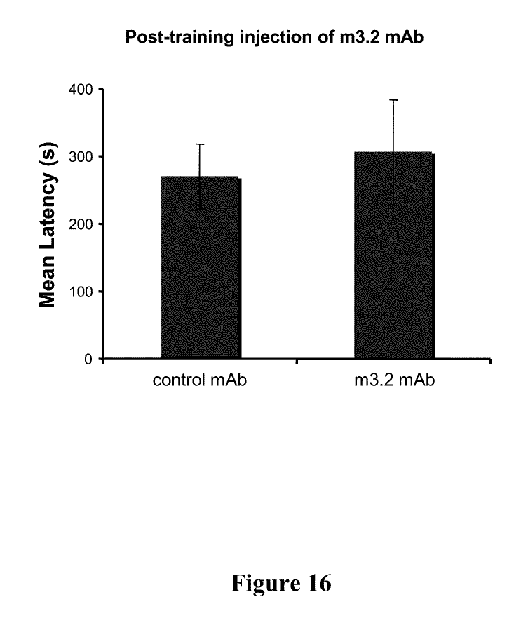

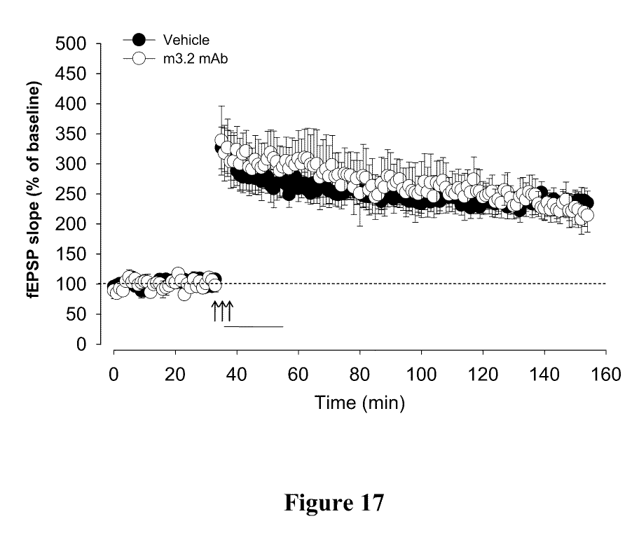

Electrophysiological measurements. 400 nm brain slices from C57B16 mice were cut and maintained in an interface chamber at 29° C. for 90 min prior to recording, as previously reported (Vitolo et al., 2002). The bath solution consisted of 124.0 mM NaCl, 4.4 mM KCl, 1.0 mM Na₂HPO₄, 25.0 mM NaHCO₃, 2.0 mM CaCl₂, 2.0 mM MgSO₄, and 10.0 mM glucose. The stimulating electrode, a bipolar tungsten electrode, was placed at the level of the Schaeffer collateral fibers whereas the recording electrode, a glass electrode filled with bath solution, was placed at the level of CA1 stratum radiatum. Basal synaptic transmission (BST) was assayed by plotting the stimulus voltages against slopes of fEPSP. Both α7-KO- and APP-KOs did not display differences in BST compared to their WT littermates (FIG. 14). For LTP experiments, a 15 min baseline was recorded every min at an intensity that evokes a response ˜35% of the maximum evoked response. LTP was induced using θ-burst stimulation (4 pulses at 100 Hz, with the bursts repeated at 5 Hz and each tetanus including 3 ten-burst trains separated by 15 sec). For PTP measurements, 3 ten-burst trains similar to those used to produce LTP were applied in the presence of 50 μM D-APV. MCL and α-BgTx were purchased from Sigma (MO). Patch clamp recordings were performed as previously described (Arancio et al., 1994). Briefly, 400 μm slices were cut with a vibratome and maintained in a submerged chamber at 29° C., perfused with artificial cerebro-spinal fluid containing 125 mM NaCl, 2.5 mM KCl, 1.25 mM Na₂HPO₄, 25 mM NaHCO₃, 2 mM CaCl₂, 1.4 mM MgCl₂, 25 mM glucose, pH=7.4 (95% O₂, 5% CO₂). Slices were permitted to recover from cutting for at least 90 min before recordings. For recordings, neurons were voltage-clamped throughout the experiment. Patch pipettes (4-6 MΩ) were pulled from thick-walled borosilicate glass tubing and filled with a solution containing 117.5 mM Cs-methylsulphonate, 17.5 mM CsCl₂, 4 mM NaCl, 0.1 mM EGTA, 10 mM HEPES, 5 mM QX-314•Cl, 4 mM MgATP, 0.3 mM Na₂GTP, 10 mM Phosphocreatine-Tris, pH adjusted to 7.3 with CsOH, osmolarity adjusted to 290 mosmoll⁻¹ with sucrose. Currents were recorded with a Warner amplifier (PC-501A), and filtered at 1 kHz (holding potential: −60 mV). The input resistance was determined from the current at the end of a 5 mV, 10 msec hyperpolarization voltage step. Synaptic input was evoked by Schaeffer collateral pathway stimulation of 150 μs pulses at a frequency of 0.1 Hz using concentric bipolar electrodes. 15 min of stable access resistance was required before beginning measurement of the EPSC amplitude. The amplitude was measured automatically by a computer using the P clamp program (version 9.2) from Axon instruments. In order to eliminate artifacts due to variation of the seal properties, the access resistance was monitored for constancy throughout all experiments.

Mice. All the mice were maintained on a 12 h light/dark cycle (with lights on at 6:00 A.M.) in temperature- and humidity-controlled rooms. Food and water were available ad libitum. Animals were killed by cervical dislocation followed by decapitation. 3-month-old male WT mice (C57BL/6) were obtained from a breeding colony kept in the animal facility of Columbia University. α7-KO mice (Orr-Urtreger et al., 1997) and their WT littermates were obtained by crossing heterozygous animals purchased from Jackson Laboratories (Bar Harbor, Me.). Mice from the 7-null mutation line were genotyped as follows: 2-mm tails from the heterozygous breedings were digested and the DNA extracted using Lysis Buffer prepared as follows: Tris-HCL 1M, EDTA 0.5M, 10% SDS, NaC15M, proteinase K in dH₂O. Jackson Laboratories supplied the sequence of primers used to identify either the neo-cassette of the null mutation or the wild-type allele, for use with the polymerase chain reaction (PCR): forward, 5′CCTGGTCCTGCTGTGTTAAACTGCTTC-3′ (SEQ ID NO:29); reverse WT(α7+), 5′-CTGCTGGGAAATCCTAGGCACACTTGAG-3′ (SEQ ID NO:30); reverse Neo(α7−), 5′-GACAAGACCGGCTTCCATCC-3′ (SEQ ID NO:31). Thermocycling conditions were as follows: 95° C. for 4 min; 35 cycles of 5° C. for 30 sec, 56° C. for 60 sec, 72° C. for 90 sec; 72° C. for 10 min; store at 4° C. PCR products were run on a 2% agarose gel, using ethidium bromide ultraviolet (UV) detection of bands at 440 by (α7+) or 750 by (α7−). APP-KO mice were obtained from a breeding colony kept in the animal facility of Nathan Kline Institute.

Infusion technique in mice. Following anaesthesia with 20 mg/kg Avertin, mice were implanted with a 26-gauge guide cannula into the dorsal part of the hippocampi (coordinates: P=2.4 mm, L=1.5 mm to a depth of 1.3 mm) (Paxinos, 1998). The cannulas were fixed to the skull with acrylic dental cement (Paladur). After 6-8 days we bilaterally injected m3.2 mAb, control mAb, Aβ₄₂, or scrambled Aβ₄₂ in a volume of 1 μl over 1 min through infusion cannulas that were connected to a microsyringe by a polyethylene tube. During infusion animals were handled gently to minimize stress. After infusion, the needle was left in place for another minute to allow diffusion. After behavioral testing, a solution of 4% methylene blue was infused into the cannulas. Animals were sacrificed and their brains were removed, frozen, and then cut at −20° with cryostat for histological localization of infusion cannulas.

Behavioral studies in mice. Contextual and cued fear conditioning were performed as previously described (Gong et al., 2004). Mice were placed in a conditioning chamber for 2 min before the onset of a tone (CS) (a 30 s, 85 dB sound at 2800 Hz). In the last 2 s of the CS, mice were given a 2 s, 0.45 mA foot shock (US) through the bars of the floor. Then, the mice were left in the conditioning chamber for another 30 s. Freezing behavior, defined as the absence of movement except for that needed for breathing, was scored using the Freezeview software. Contextual fear learning was evaluated 24 hrs after training by measuring freezing for 5 min in the chamber in which the mice were trained. Cued fear learning was evaluated 24 hrs after contextual testing by placing mice in a novel context for 2 min (pre-CS test), after which they were exposed to the CS for 3 min (CS test), and freezing was measured. Sensory perception of the shock was determined through threshold assessment, as described (Gong et al., 2004). Briefly, the electric current (0.1 mA for 1 s) was increased at 30 s intervals by 0.1 mA to 0.7 mA. Threshold to flinching (first visible response to shock), jumping (first extreme motor response), and screaming (first vocalized distress) was quantified for each animal by averaging of the shock intensity at which each animal manifested a behavioral response of that type to the foot shock. Visual, motor, and motivation skills were also tested using a visible platform to measure the time and the speed to reach a visible platform placed within a pool filled with water by means of a video tracking system (HVS-2020, HVS Image, UK) (Gong et al., 2004). No difference in the sensory threshold assessment as well as in the time and the speed to reach the platform was observed among different groups of mice in experiments in which fear conditioning was tested.

Statistical Analyses. Animals were always coded to blind investigators with respect to treatment. Results are expressed as Standard Error of the Mean (SEM). Level of significance is set for p<0.05. For experiments on mice data were analyzed by Student's t test (pairwise comparisons) or two-way ANOVA for time and treatment with repeated measures (multiple comparisons). For experiments on rats results were analyzed with ANOVA with post-hoc correction with treatment condition as main effect followed by a Newman-Keuls post-hoc t test.

While the foregoing invention has been described in some detail for purposes of clarity and understanding, these particular embodiments and examples are to be considered as illustrative and not restrictive. It will be appreciated by one skilled in the art from a reading of this disclosure that various changes in form and detail can be made without departing from the true scope of the invention.

### REFERENCES

1. Cullen, W. K., Y. H. Suh, R. Anwyl, and M. J. Rowan, Block of LTP in rat hippocampus in vivo by beta-amyloid precursor protein fragments. Neuroreport, 1997. 8(15): p. 3213-7.
2. Itoh, A., T. Akaike, M. Sokabe, A. Nitta, R. Iida, A. Olariu, K. Yamada, and T. Nabeshima, Impairments of long-term potentiation in hippocampal slices of beta-amyloid-infused rats. Eur J Pharmacol, 1999. 382(3): p. 167-75.
3. Walsh, D. M., I. Klyubin, J. V. Fadeeva, W. K. Cullen, R. Anwyl, M. S. Wolfe, M. J. Rowan, and D. J. Selkoe, Naturally secreted oligomers of amyloid beta protein potently inhibit hippocampal long-term potentiation in vivo. Nature, 2002. 416(6880): p. 535-9.
4. Vitolo, O. V., A. Sant'Angelo, V. Costanzo, F. Battaglia, O. Arancio, and M. Shelanski, Amyloid beta-peptide inhibition of the PKA/CREB pathway and long-term potentiation: reversibility by drugs that enhance cAMP signaling. Proc Natl Acad Sci USA, 2002. 99(20): p. 13217-21.
5. Puzzo, D., O. Vitolo, F. Trinchese, J. P. Jacob, A. Palmeri, and O. Arancio, Amyloid-beta peptide inhibits activation of the nitric oxide/cGMP/cAMP-responsive element-binding protein pathway during hippocampal synaptic plasticity. J Neurosci, 2005. 25(29): p. 6887-97.
6. Klyubin, I., D. M. Walsh, C. A. Lernere, W. K. Cullen, G. M. Shankar, V. Betts, E. T. Spooner, L. Jiang, R. Anwyl, D. J. Selkoe, and M. J. Rowan, Amyloid beta protein immunotherapy neutralizes Abeta oligomers that disrupt synaptic plasticity in vivo. Nat Med, 2005. 11(5): p. 556-61.
7. Glenner, G. G. and C. W. Wong, Alzheimer's disease: initial report of the purification and characterization of a novel cerebrovascular amyloid protein. Biochem Biophys Res Commun, 1984. 120(3): p. 885-90.
8. Freir, D. B., C. Holscher, and C. E. Herron, Blockade of long-term potentiation by beta-amyloid peptides in the CA1 region of the rat hippocampus in vivo. J Neurophysiol, 2001. 85(2): p. 708-13.
9. Kim, J. H., R. Anwyl, Y. H. Suh, M. B. Djamgoz, and M. J. Rowan, Use-dependent effects of amyloidogenic fragments of (beta)-amyloid precursor protein on synaptic plasticity in rat hippocampus in vivo. J Neurosci, 2001. 21(4): p. 1327-33.
10. Stephan, A., S. Laroche, and S. Davis, Generation of aggregated beta-amyloid in the rat hippocampus impairs synaptic transmission and plasticity and causes memory deficits. J Neurosci, 2001. 21(15): p. 5703-14.
11. Sant'Angelo, A., F. Trinchese, and O. Arancio, Usefulness of behavioral and electrophysiological studies in transgenic models of Alzheimer's disease. Neurochem Res, 2003. 28(7): p. 1009-15.
12. Malm, T., M. Ort, L. Tahtivaara, N. Jukarainen, G. Goldsteins, J. Puolivali, A. Nurmi, R. Pussinen, T. Ahtoniemi, T. K. Miettinen, K. Kanninen, S. Leskinen, N. Vartiainen, J. Yrjanheikki, R. Laatikainen, M. E. Harris-White, M. Koistinaho, S. A. Frautschy, J. Bures, and J. Koistinaho, beta-Amyloid infusion results in delayed and age-dependent learning deficits without role of inflammation or beta-amyloid deposits. Proc Natl Acad Sci USA, 2006. 103(23): p. 8852-7.
13. Cleary, J. P., D. M. Walsh, J. J. Hofmeister, G. M. Shankar, M. A. Kuskowski, D. J. Selkoe, and K. H. Ashe, Natural oligomers of the amyloid-beta protein specifically disrupt cognitive function. Nat Neurosci, 2005. 8(1): p. 79-84.
14. Cirrito, J. R., P. C. May, M. A. O'Dell, J. W. Taylor, M. Parsadanian, J. W. Cramer, J. E. Audia, J. S, Nissen, K. R. Bales, S. M. Paul, R. B. DeMattos, and D. M. Holtzman, In vivo assessment of brain interstitial fluid with microdialysis reveals plaque-associated changes in amyloid-beta metabolism and half-life. J Neurosci, 2003. 23(26): p. 8844-53.
15. Bliss, T. V. and G. L. Collingridge, A synaptic model of memory: long-term potentiation in the hippocampus. Nature, 1993. 361(6407): p. 31-9.
16. Hardy, J. and D. J. Selkoe, The amyloid hypothesis of Alzheimer's disease: progress and problems on the road to therapeutics. Science, 2002. 297(5580): p. 353-6.
17. Buee, L., T. Bussiere, V. Buee-Scherrer, A. Delacourte, and P. R. H of, Tau protein isoforms, phosphorylation and role in neurodegenerative disorders. Brain Res Brain Res Rev, 2000. 33(1): p. 95-130.
18. Goldgaber, D., M. I. Lerman, O. W. McBride, U. Saffiotti, and D. C. Gajdusek, Characterization and chromosomal localization of a cDNA encoding brain amyloid of Alzheimer's disease. Science, 1987. 235(4791): p. 877-80.
19. Kang, J., H. G. Lemaire, A. Unterbeck, J. M. Salbaum, C. L. Masters, K. H. Grzeschik, G. Multhaup, K. Beyreuther, and B. Muller-Hill, The precursor of Alzheimer's disease amyloid A4 protein resembles a cell-surface receptor. Nature, 1987. 325(6106): p. 733-6.
20. Kitaguchi, N., Y. Takahashi, Y. Tokushima, S. Shiojiri, and H. Ito, Novel precursor of Alzheimer's disease amyloid protein shows protease inhibitory activity. Nature, 1988. 331(6156): p. 530-2.
21. Robakis, N. K., N. Ramakrishna, G. Wolfe, and H. M. Wisniewski, Molecular cloning and characterization of a cDNA encoding the cerebrovascular and the neuritic plaque amyloid peptides. Proc Natl Acad Sci USA, 1987. 84(12): p. 4190-4.
22. Tanzi, R. E., J. F. Gusella, P. C. Watkins, G. A. Bruns, P. St George-Hyslop, M. L. Van Keuren, D. Patterson, S. Pagan, D. M. Kurnit, and R. L. Neve, Amyloid beta protein gene: cDNA, mRNA distribution, and genetic linkage near the Alzheimer locus. Science, 1987. 235(4791): p. 880-4.
23. Vetrivel, K. S. and G. Thinakaran, Amyloidogenic processing of beta-amyloid precursor protein in intracellular compartments. Neurology, 2006. 66(2 Suppl 1): p. S69-73.
24. De Strooper, B. and W. Annaert, Proteolytic processing and cell biological functions of the amyloid precursor protein. J Cell Sci, 2000. 113 (Pt 11): p. 1857-70.
25. Mattson, M. P., Cellular actions of beta-amyloid precursor protein and its soluble and fibrillogenic derivatives. Physiol Rev, 1997. 77(4): p. 1081-132.
26. Smith-Swintosky, V. L., L. C. Pettigrew, S. D. Craddock, A. R. Culwell, R. E. Rydel, and M. P. Mattson, Secreted forms of beta-amyloid precursor protein protect against ischemic brain injury. J Neurochem, 1994. 63(2): p. 781-4.
27. Mattson, M. P., Secreted forms of beta-amyloid precursor protein modulate dendrite outgrowth and calcium responses to glutamate in cultured embryonic hippocampal neurons. J Neurobiol, 1994. 25(4): p. 439-50.
28. Araki, W., N. Kitaguchi, Y. Tokushima, K. Ishii, H. Aratake, S. Shimohama, S. Nakamura, and J. Kimura, Trophic effect of beta-amyloid precursor protein on cerebral cortical neurons in culture. Biochem Biophys Res Commun, 1991. 181(1): p. 265-71.
29. Furukawa, K., B. L. Sopher, R. E. Rydel, J. G. Begley, D. G. Pham, G. M. Martin, M. Fox, and M. P. Mattson, Increased activity-regulating and neuroprotective efficacy of alpha-secretase-derived secreted amyloid precursor protein conferred by a C-terminal heparin-binding domain. J Neurochem, 1996. 67(5): p. 1882-96.
30. Mucke, L., E. Masliah, W. B. Johnson, M. D. Ruppe, M. Alford, E. M. Rockenstein, S. Forss-Petter, M. Pietropaolo, M. Mallory, and C. R. Abraham, Synaptotrophic effects of human amyloid beta protein precursors in the cortex of transgenic mice. Brain Res, 1994. 666(2): p. 151-67.
31. Gao, Y. and S. W. Pimplikar, The gamma-secretase-cleaved C-terminal fragment of amyloid precursor protein mediates signaling to the nucleus. Proc Natl Acad Sci USA, 2001. 98(26): p. 14979-84.
32. Kimberly, W. T., J. B. Zheng, S. Y. Guenette, and D. J. Selkoe, The intracellular domain of the beta-amyloid precursor protein is stabilized by Fe65 and translocates to the nucleus in a notch-like manner. J Biol Chem, 2001. 276(43): p. 40288-92.
33. Cao, X. and T. C. Sudhof, A transcriptionally [correction of transcriptively] active complex of APP with Fe65 and histone acetyltransferase Tip60. Science, 2001. 293(5527): p. 115-20.
34. Rocchi, A., S. Pellegrini, G. Siciliano, and L. Murri, Causative and susceptibility genes for Alzheimer's disease: a review. Brain Res Bull, 2003. 61(1): p. 1-24.
35. Salbaum, J. M. and F. H. Ruddle, Embryonic expression pattern of amyloid protein precursor suggests a role in differentiation of specific subsets of neurons. J Exp Zool, 1994. 269(2): p. 116-27.
36. Qiu, W. Q., A. Ferreira, C. Miller, E. H. Koo, and D. J. Selkoe, Cell-surface beta-amyloid precursor protein stimulates neurite outgrowth of hippocampal neurons in an isoform-dependent manner. J Neurosci, 1995. 15(3 Pt 2): p. 2157-67.
37. Huber, G., J. R. Martin, J. Loffler, and J. L. Moreau, Involvement of amyloid precursor protein in memory formation in the rat: an indirect antibody approach. Brain Res, 1993. 603(2): p. 348-52.
38. Ishida, A., K. Furukawa, J. N. Keller, and M. P. Mattson, Secreted form of beta-amyloid precursor protein shifts the frequency dependency for induction of LTD, and enhances LTP in hippocampal slices. Neuroreport, 1997. 8(9-10): p. 2133-7.
39. Dawson, G. R., G. R. Seabrook, H. Zheng, D. W. Smith, S. Graham, G. O'Dowd, B. J. Bowery, S. Boyce, M. E. Trumbauer, H. Y. Chen, L. H. Van der Ploeg, and D. J. Sirinathsinghji, Age-related cognitive deficits, impaired long-term potentiation and reduction in synaptic marker density in mice lacking the beta-amyloid precursor protein. Neuroscience, 1999. 90(1): p. 1-13.
40. Seabrook, G. R., D. W. Smith, B. J. Bowery, A. Easter, T. Reynolds, S. M. Fitzjohn, R. A. Morton, H. Zheng, G. R. Dawson, D. J. Sirinathsinghji, C. H. Davies, G. L. Collingridge, and R. G. Hill, Mechanisms contributing to the deficits in hippocampal synaptic plasticity in mice lacking amyloid precursor protein. Neuropharmacology, 1999. 38(3): p. 349-59.
41. Phinney, A. L., M. E. Calhoun, D. P. Wolfer, H. P. Lipp, H. Zheng, and M. Jucker, No hippocampal neuron or synaptic bouton loss in learning-impaired aged beta-amyloid precursor protein-null mice. Neuroscience, 1999. 90(4): p. 1207-16.
42. Wasco, W., K. Bupp, M. Magendantz, J. F. Gusella, R. E. Tanzi, and F. Solomon, Identification of a mouse brain cDNA that encodes a protein related to the Alzheimer disease-associated amyloid beta protein precursor. Proc Natl Acad Sci USA, 1992. 89(22): p. 10758-62.
43. Wasco, W., S. Gurubhagavatula, M. D. Paradis, D. M. Romano, S. S. Sisodia, B. T. Hyman, R. L. Neve, and R. E. Tanzi, Isolation and characterization of APLP2 encoding a homologue of the Alzheimer's associated amyloid beta protein precursor. Nat Genet, 1993. 5(1): p. 95-100.
44. Slunt, H. H., G. Thinakaran, C. Von Koch, A. C. Lo, R. E. Tanzi, and S. S. Sisodia, Expression of a ubiquitous, cross-reactive homologue of the mouse beta-amyloid precursor protein (APP). J Biol Chem, 1994. 269(4): p. 2637-44.
45. Cirrito, J. R., K. A. Yamada, M. B. Finn, R. S. Sloviter, K. R. Bales, P. C. May, D. D. Schoepp, S. M. Paul, S. Mennerick, and D. M. Holtzman, Synaptic activity regulates interstitial fluid amyloid-beta levels in vivo. Neuron, 2005. 48(6): p. 913-22.
46. Kamenetz, F., T. Tomita, H. Hsieh, G. Seabrook, D. Borchelt, T. Iwatsubo, S. Sisodia, and R. Malinow, APP processing and synaptic function. Neuron, 2003. 37(6): p. 925-37.
47. Selkoe, D. and R. Kopan, Notch and Presenilin: regulated intramembrane proteolysis links development and degeneration. Annu Rev Neurosci, 2003. 26: p. 565-97.
48. Saura, C. A., S. Y. Choi, V. Beglopoulos, S. Malkani, D. Zhang, B. S. Shankaranarayana Rao, S. Chattarji, R. J. Kelleher, 3rd, E. R. Kandel, K. Duff, A. Kirkwood, and J. Shen, Loss of presenilin function causes impairments of memory and synaptic plasticity followed by age-dependent neurodegeneration. Neuron, 2004. 42(1): p. 23-36.
49. Dewachter, I., L. R is, S. Croes, P. Borghgraef, T. Voets, B. Nilius, E. Godaux, and F. Van Leuven. Differential affects of presenilin-1 deficiency and mutant presenilin-1 on synaptic activity. in Alzheimer's Association 10th International Conference on Alzheimer's Disease and Related Disorders. 2006. Madrid, Spain.
50. Nakagami, Y., S, Nishimura, T. Murasugi, I. Kaneko, M. Meguro, S. Marumoto, H. Kogen, K. Koyama, and T. Oda, A novel beta-sheet breaker, RS-0406, reverses amyloid beta-induced cytotoxicity and impairment of long-term potentiation in vitro. Br J Pharmacol, 2002. 137(5): p. 676-82.
51. Walsh, D. M., M. Townsend, M. B. Podlisny, G. M. Shankar, J. V. Fadeeva, O. E. Agnaf, D. M. Hartley, and D. J. Selkoe, Certain inhibitors of synthetic amyloid beta-peptide (Abeta) fibrillogenesis block oligomerization of natural Abeta and thereby rescue long-term potentiation. J Neurosci, 2005. 25(10): p. 2455-62.
52. Schenk, D., R. Barbour, W. Dunn, G. Gordon, H. Grajeda, T. Guido, K. Hu, J. Huang, K. Johnson-Wood, K. Khan, D. Kholodenko, M. Lee, Z. Liao, I. Lieberburg, R. Motter, L. Mutter, F. Soriano, G. Shopp, N. Vasquez, C. Vandevert, S. Walker, M. Wogulis, T. Yednock, D. Games, and P. Seubert, Immunization with amyloid-beta attenuates Alzheimer-disease-like pathology in the PDAPP mouse. Nature, 1999. 400(6740): p. 173-7.
53. Wu, J., R. Anwyl, and M. J. Rowan, beta-Amyloid-(1-40) increases long-term potentiation in rat hippocampus in vitro. Eur J Pharmacol, 1995. 284(3): p. R1-3.
54. Kowalska, M. A. and K. Badellino, beta-Amyloid protein induces platelet aggregation and supports platelet adhesion. Biochem Biophys Res Commun, 1994. 205(3): p. 1829-35.
55. Mattson, M. P., Z. H. Guo, and J. D. Geiger, Secreted form of amyloid precursor protein enhances basal glucose and glutamate transport and protects against oxidative impairment of glucose and glutamate transport in synaptosomes by a cyclic GMP-mediated mechanism. J Neurochem, 1999. 73(2): p. 532-7.
56. Malin, D. H., M. K. Crothers, J. R. Lake, P. Goyarzu, R. E. Plotner, S. A. Garcia, S. H. Spell, B. J. Tomsic, T. Giordano, and N. W. Kowall, Hippocampal injections of amyloid beta-peptide 1-40 impair subsequent one-trial/day reward learning. Neurobiol Learn Mem, 2001. 76(2): p. 125-37.
57. Mathews, P. M., A. M. Cataldo, B. H. Kao, A. G. Rudnicki, X. Qin, J. L. Yang, Y. Jiang, M. Picciano, C. Hulette, C. F. Lippa, T. D. Bird, D. Nochlin, J. Walter, C. Haass, L. Levesque, P. E. Fraser, A. Andreadis, and R. A. Nixon, Brain expression of presenilins in sporadic and early-onset, familial Alzheimer's disease. Mol Med, 2000. 6(10): p. 878-91.
58. Herzig, M. C., D. T. Winkler, P. Burgermeister, M. Pfeifer, E. Kohler, S. D. Schmidt, S. Danner, D. Abramowski, C. Sturchler-Pierrat, K. Burki, S. G. van Duinen, M. L. Maat-Schieman, M. Staufenbiel, P. M. Mathews, and M. Jucker, Abeta is targeted to the vasculature in a mouse model of hereditary cerebral hemorrhage with amyloidosis. Nat Neurosci, 2004. 7(9): p. 954-60.
59. Mastrangelo, P., P. M. Mathews, M. A. Chishti, S. D. Schmidt, Y. Gu, J. Yang, M. J. Mazzella, J. Coomaraswamy, P. Home, B. Strome, H. Pelly, G. Levesque, C. Ebeling, Y. Jiang, R. A. Nixon, R. Rozmahel, P. E. Fraser, P. St George-Hyslop, G. A. Carlson, and D. Westaway, Dissociated phenotypes in presenilin transgenic mice define functionally distinct gamma-secretases. Proc Natl Acad Sci USA, 2005. 102(25): p. 8972-7.
60. Pastorino, L., A. F. Ikin, S. Lamprianou, N. Vacaresse, J. P. Revelli, K. Platt, P. Paganetti, P. M. Mathews, S. Harroch, and J. D. Buxbaum, BACE (beta-secretase) modulates the processing of APLP2 in vivo. Mol Cell Neurosci, 2004. 25(4): p. 642-9.
61. Phinney, A. L., B. Drisaldi, S. D. Schmidt, S. Lugowski, V. Coronado, Y. Liang, P. Home, J. Yang, J. Sekoulidis, J. Coomaraswamy, M. A. Chishti, D. W. Cox, P. M. Mathews, R. A. Nixon, G. A. Carlson, P. St George-Hyslop, and D. Westaway, In vivo reduction of amyloid-beta by a mutant copper transporter. Proc Natl Acad Sci USA, 2003. 100(24): p. 14193-8.
62. Radde, R., T. Bolmont, S. A. Kaeser, J. Coomaraswamy, D. Lindau, L. Stoltze, M. E. Calhoun, F. Jaggi, H. Wolburg, S. Gengler, C. Haass, B. Ghetti, C. Czech, C. Holscher, P. M. Mathews, and M. Jucker, Abeta42-driven cerebral amyloidosis in transgenic mice reveals early and robust pathology. EMBO Rep, 2006. 7(9): p. 940-6.
63. Rozmahel, R., J. Huang, F. Chen, Y. Liang, V. Nguyen, M. Ikeda, G. Levesque, G. Yu, M. Nishimura, P. Mathews, S. D. Schmidt, M. Mercken, C. Bergeron, D. Westaway, and P. St George-Hyslop, Normal brain development in PS1 hypomorphic mice with markedly reduced gamma-secretase cleavage of betaAPP. Neurobiol Aging, 2002. 23(2): p. 187-94.
64. Rozmahel, R., H. T. Mount, F. Chen, V. Nguyen, J. Huang, S. Erdebil, J. Liauw, G. Yu, H. Hasegawa, Y. Gu, Y. Q. Song, S. D. Schmidt, R. A. Nixon, P. M. Mathews, C. Bergeron, P. Fraser, D. Westaway, and P. St George-Hyslop, Alleles at the Nicastrin locus modify presenilin 1-deficiency phenotype. Proc Natl Acad Sci USA, 2002. 99(22): p. 14452-7.
65. Schmidt, S. D., Y. Jiang, R. A. Nixon, and P. M. Mathews, Tissue processing prior to protein analysis and amyloid-beta quantitation. Methods Mol Biol, 2005. 299: p. 267-78.
66. Schmidt, S. D., R. A. Nixon, and P. M. Mathews, ELISA method for measurement of amyloid-beta levels. Methods Mol Biol, 2005. 299: p. 279-97.
67. Trinchese, F., S. Liu, F. Battaglia, S. Walter, P. M. Mathews, and O. Arancio, Progressive age-related development of Alzheimer-like pathology in APP/PS1 mice. Ann Neurol, 2004. 55(6): p. 801-14.
68. Yao, J., S. S. Petanceska, T. J. Montine, D. M. Holtzman, S. D. Schmidt, C. A. Parker, M. J. Callahan, W. J. Lipinski, C. L. Bisgaier, B. A. Turner, R. A. Nixon, R. N. Martins, C. Ouimet, J. D. Smith, P. Davies, E. Laska, M. E. Ehrlich, L. C. Walker, P. M. Mathews, and S. Gandy, Aging, gender and APOE isotype modulate metabolism of Alzheimer's Abeta peptides and F-isoprostanes in the absence of detectable amyloid deposits. J Neurochem, 2004. 90(4): p. 1011-8.
69. Yu, W. H., A. M. Cuervo, A. Kumar, C. M. Peterhoff, S. D. Schmidt, J. H. Lee, P. S. Mohan, M. Mercken, M. R. Farmery, L. O. Tjernberg, Y. Jiang, K. Duff, Y. Uchiyama, J. Naslund, P. M. Mathews, A. M. Cataldo, and R. A. Nixon, Macroautophagy—a novel Beta-amyloid peptide-generating pathway activated in Alzheimer's disease. J Cell Biol, 2005. 171(1): p. 87-98.
70. Pfeifer, M., S. Boncristiano, L. Bondolfi, A. Stalder, T. Deller, M. Staufenbiel, P. M. Mathews, and M. Jucker, Cerebral hemorrhage after passive anti-Abeta immunotherapy. Science, 2002. 298(5597): p. 1379.
71. Pawlik, M., M. Sastre, M. Calero, P. M. Mathews, S. D. Schmidt, R. A. Nixon, and E. Levy, Overexpression of human cystatin C in transgenic mice does not affect levels of endogenous brain amyloid Beta Peptide. J Mol Neurosci, 2004. 22(1-2): p. 13-8.
72. Schroeder, B. E. and E. H. Koo, To think or not to think: synaptic activity and Abeta release. Neuron, 2005. 48(6): p. 873-5.
73. Selkoe, D. J., Alzheimer's disease is a synaptic failure. Science, 2002. 298(5594): p. 789-91.
74. Dodart, J. C., K. R. Bales, K. S. Gannon, S. J. Greene, R. B. DeMattos, C. Mathis, C. A. DeLong, S. Wu, X. Wu, D. M. Holtzman, and S. M. Paul, Immunization reverses memory deficits without reducing brain Abeta burden in Alzheimer's disease model. Nat Neurosci, 2002. 5(5): p. 452-7.
75. Janus, C., J. Pearson, J. McLaurin, P. M. Mathews, Y. Jiang, S. D. Schmidt, M. A. Chishti, P. Home, D. Heslin, J. French, H. T. Mount, R. A. Nixon, M. Mercken, C. Bergeron, P. E. Fraser, P. St George-Hyslop, and D. Westaway, A beta peptide immunization reduces behavioural impairment and plaques in a model of Alzheimer's disease. Nature, 2000. 408(6815): p. 979-82.
76. Morgan, D., D. M. Diamond, P. E. Gottschall, K. E. Ugen, C. Dickey, J. Hardy, K. Duff, P. Jantzen, G. DiCarlo, D. Wilcock, K. Connor, J. Hatcher, C. Hope, M. Gordon, and G. W. Arendash, A beta peptide vaccination prevents memory loss in an animal model of Alzheimer's disease. Nature, 2000. 408(6815): p. 982-5.
77. Barghorn, S., V. Nimmrich, A. Striebinger, C. Krantz, P. Keller, B. Janson, M. Bahr, M. Schmidt, R. S. Bitner, J. Harlan, E. Barlow, U. Ebert, and H. Hillen, Globular amyloid beta-peptide oligomer—a homogenous and stable neuropathological protein in Alzheimer's disease. J Neurochem, 2005. 95(3): p. 834-47.
78. Lesne, S., M. T. Koh, L. Kotilinek, R. Kayed, C. G. Glabe, A. Yang, M. Gallagher, and K. H. Ashe, A specific amyloid-beta protein assembly in the brain impairs memory. Nature, 2006. 440(7082): p. 352-7.
79. Jarrett, J. T., E. P. Berger, and P. T. Lansbury, Jr., The C-terminus of the beta protein is critical in amyloidogenesis. Ann N Y Acad Sci, 1993. 695: p. 144-8.
80. Petrone, A., F. Battaglia, C. Wang, A. Dusa, J. Su, D. Zagzag, R. Bianchi, P. Casaccia-Bonnefil, O. Arancio, and J. Sap, Receptor protein tyrosine phosphatase alpha is essential for hippocampal neuronal migration and long-term potentiation. Embo J, 2003. 22(16): p. 4121-31.
81. Gong, B., Z. Cao, P. Zheng, O. V. Vitolo, S. Liu, A. Staniszewski, D. Moolman, H. Zhang, M. Shelanski, and O. Arancio, Ubiquitin Hydrolase Uch-L1 Rescues beta-Amyloid-Induced Decreases in Synaptic Function and Contextual Memory. Cell, 2006. 126(4): p. 775-88.
82. Gong, B., O. V. Vitolo, F. Trinchese, S. Liu, M. Shelanski, and O. Arancio, Persistent improvement in synaptic and cognitive functions in an Alzheimer mouse model following rolipram treatment. J. Clin. Invest., 2004. 114: p. 1624-1634.
83. Arancio, O., H. Korn, A. Gulyas, T. Freund, and R. Miles, Excitatory synaptic connections onto rat hippocampal inhibitory cells may involve a single transmitter release site. J Physiol, 1994. 481 (Pt 2): p. 395-405.
84. Moretti, P., J. M. Levenson, F. Battaglia, R. Atkinson, R. Teague, B. Antalffy, D. Armstrong, O. Arancio, J. D. Sweatt, and H. Y. Zoghbi, Learning and memory and synaptic plasticity are impaired in a mouse model of Rett syndrome. J Neurosci, 2006. 26(1): p. 319-27.
85. Yano, H., I. Ninan, H. Zhang, T. A. Milner, O. Arancio, and M. V. Chao, BDNF-mediated neurotransmission relies upon a myosin VI motor complex. Nat Neurosci, 2006. 9(8): p. 1009-18.
86. MacDermott, A. B., D. Reichling, and O. Arancio, Mechanisms underlying excitatory amino acid-evoked calcium entry in cultured neurons from the embryonic rat spinal cord. Advances in Experimental Medicine and Biology, Excitatory Amino Acids and Neuronal Plasticity. 1990: Plenum Press. 117-124.
87. Taubenfeld, S. M., M. H. Milekic, B. Monti, and C. M. Alberini, The consolidation of new but not reactivated memory requires hippocampal C/EBPbeta. Nat Neurosci, 2001. 4(8): p. 813-8.
88. Garcia-Osta, A., P. Tsokas, G. Pollonini, E. M. Landau, R. Blitzer, and C. M. Alberini, MuSK expressed in the brain mediates cholinergic responses, synaptic plasticity, and memory formation. J Neurosci, 2006. 26(30): p. 7919-32.
89. Tronel, S., M. H. Milekic, and C. M. Alberini, Linking new information to a reactivated memory requires consolidation and not reconsolidation mechanisms. PLoS Biol, 2005. 3(9): p. e293.
90. Paxinos, G., Mouse brain in stereotaxic coordinates. 2nd ed. 1998, New York: Academic Press.
91. Izquierdo, I. and J. H. Medina, Memory formation: the sequence of biochemical events in the hippocampus and its connection to activity in other brain structures. Neurobiol Learn Mem, 1997. 68(3): p. 285-316.
92. Ambrogi Lorenzini, C. G., E. Baldi, C. Bucherelli, B. Sacchetti, and G. Tassoni, Neural topography and chronology of memory consolidation: a review of functional inactivation findings. Neurobiol Learn Mem, 1999. 71(1): p. 1-18.
93. McGaugh, J. L., Memory consolidation and the amygdala: a systems perspective. Trends Neurosci, 2002. 25(9): p. 456.
94. Sacchetti, B., C. A. Lorenzini, E. Baldi, C. Bucherelli, M. Roberto, G. Tassoni, and M. Brunelli, Long-lasting hippocampal potentiation and contextual memory consolidation. Eur J Neurosci, 2001. 13(12): p. 2291-8.
95. Izquierdo, L. A., D. M. Barros, M. R. Vianna, A. Coitinho, T. deDavid e Silva, H. Choi, B. Moletta, J. H. Medina, and I. Izquierdo, Molecular pharmacological dissection of short- and long-term memory. Cell Mol Neurobiol, 2002. 22(3): p. 269-87.
96. Kudryashova, I. V., Responses of hippocampal neurons at different stages of acquisition of conditioned reflex avoidance in rats. Neurosci Behav Physiol, 2002. 32(2): p. 121-7.
97. Cammarota, M., L. R. Bevilaqua, D. M. Barros, M. R. Vianna, L. A. Izquierdo, J. H. Medina, and I. Izquierdo, Retrieval and the extinction of memory. Cell Mol Neurobiol, 2005. 25(3-4): p. 465-74.
98. Alberini, C. M., M. H. Milekic, and S. Tronel, Mechanisms of memory stabilization and de-stabilization. Cell Mol Life Sci, 2006. 63(9): p. 999-1008.
99. Alberini, C. M., S. M. Taubenfeld, and A. Garcia-Osta, CREB and the CREB-C/EBP-dependent gene expression cascade in long-term memory. Cellscience Reviews, 2005. 2(2).
100. Stine, W. B., Jr., K. N. Dahlgren, G. A. Krafft, and M. J. LaDu, In vitro characterization of conditions for amyloid-beta peptide oligomerization and fibrillogenesis. J Biol Chem, 2003. 278(13): p. 11612-22.
101. Bitan, G., M. D. Kirkitadze, A. Lomakin, S. S. Vollers, G. B. Benedek, and D. B. Teplow, Amyloid beta-protein (Abeta) assembly: Abeta 40 and Abeta 42 oligomerize through distinct pathways. Proc Natl Acad Sci USA, 2003. 100(1): p. 330-5.
102. Schagger, H. and G. von Jagow, Tricine-sodium dodecyl sulfate-polyacrylamide gel electrophoresis for the separation of proteins in the range from 1 to 100 kDa. Anal Biochem, 1987. 166(2): p. 368-79.
103. Grbovic, O. M., P. M. Mathews, Y. Jiang, S. D. Schmidt, R. Dinakar, N B Summers-Terio, B. P. Ceresa, R. A. Nixon, and A. M. Cataldo, RabS-stimulated up-regulation of the endocytic pathway increases intracellular beta-cleaved amyloid precursor protein carboxyl-terminal fragment levels and Abeta production. J Biol Chem, 2003. 278(33): p. 31261-8.
104. Mathews, P. M., C. B. Guerra, Y. Jiang, O. M. Grbovic, B. H. Kao, S. D. Schmidt, R. Dinakar, M. Mercken, A. Hille-Rehfeld, J. Rohrer, P. Mehta, A. M. Cataldo, and R. A. Nixon, Alzheimer's disease-related overexpression of the cation-dependent mannose 6-phosphate receptor increases Abeta secretion: role for altered lysosomal hydrolase distribution in beta-amyloidogenesis. J Biol Chem, 2002. 277(7): p. 5299-307.
105. Mathews, P. M., Y. Jiang, S. D. Schmidt, O. M. Grbovic, M. Mercken, and R. A. Nixon, Calpain activity regulates the cell surface distribution of amyloid precursor protein. Inhibition of clapains enhances endosomal generation of beta-cleaved C-terminal APP fragments. J Biol Chem, 2002. 277(39): p. 36415-24.
106. Leznik, E., M. Fa', and O. Arancio. Long-term potentiation enhances SUMOylation in the CA1 region of hippocampus. in Soc. Neurosci. Abstr. 2006. Atlanta (USA).
107. Schmidt, S. D., Y. Jiang, S. P. Jacobsen, M. Mercken, R. A. Nixon, and P. M. Mathews. Detection of mouse Ab in wild-type and b-amyloid depositing transgenic mice: improved methods for the detection of multiple APP metabolites. in Society for Neuroscience Annual Meeting. 2001.
108. Meyer-Luehmann, M., M. Stalder, M. C. Herzig, S. A. Kaeser, E. Kohler, M. Pfeifer, S. Boncristiano, P. M. Mathews, M. Mercken, D. Abramowski, M. Staufenbiel, and M. Jucker, Extracellular amyloid formation and associated pathology in neural grafts. Nat Neurosci, 2003. 6(4): p. 370-7.
109. Goebel, S. M., R. M. Alvestad, S. J. Coultrap, and M. D. Browning, Tyrosine phosphorylation of the N-methyl-D-aspartate receptor is enhanced in synaptic membrane fractions of the adult rat hippocampus. Brain Res Mol Brain Res, 2005. 142(1): p. 65-79.
110. Lu, W. Y., Z. G. Xiong, S. Lei, B. A. Orser, E. Dudek, M. D. Browning, and J. F. MacDonald, G-protein-coupled receptors act via protein kinase C and Src to regulate NMDA receptors. Nat Neurosci, 1999. 2(4): p. 331-8.
111. Wu, J., R. Anwyl, and M. J. Rowan, beta-Amyloid selectively augments NMDA receptor-mediated synaptic transmission in rat hippocampus. Neuroreport, 1995. 62409-13(17): p. 2409-13.
112. Snyder, E. M., Y. Nong, C. G. Almeida, S. Paul, T. Moran, E. Y. Choi, A. C. Nairn, M. W. Salter, P. J. Lombroso, G. K. Gouras, and P. Greengard, Regulation of NMDA receptor trafficking by amyloid-beta. Nat Neurosci, 2005. 8(8): p. 1051-8.
113. Scheetz, A. J., A. C. Nairn, and M. Constantine-Paton, NMDA receptor-mediated control of protein synthesis at developing synapses. Nat Neurosci, 2000. 3(3): p. 211-6.
114. Grosshans, D. R., D. A. Clayton, S. J. Coultrap, and M. D. Browning, LTP leads to rapid surface expression of NMDA but not AMPA receptors in adult rat CA1. Nat Neurosci, 2002. 5(1): p. 27-33.
115. Dunah, A. W. and D. G. Standaert, Dopamine D1 receptor-dependent trafficking of striatal NMDA glutamate receptors to the postsynaptic membrane. J Neurosci, 2001. 21(15): p. 5546-58.
116. Taubenfeld, S. M., K. A. Wiig, B. Monti, B. Dolan, G. Pollonini, and C. M. Alberini, Formix-dependent induction of hippocampal CCAAT enhancer-binding protein [beta] and [delta] Co-localizes with phosphorylated cAMP response element-binding protein and accompanies long-term memory consolidation. J Neurosci, 2001. 21(1): p. 84-91.
117. Levin, E. D. and B. B. Simon, Nicotinic acetylcholine involvement in cognitive function in animals. Psychopharmacology (Berl), 1998. 138(3-4): p. 217-30.
118. Jones, S., S. Sudweeks, and J. L. Yakel, Nicotinic receptors in the brain: correlating physiology with function. Trends Neurosci, 1999. 22(12): p. 555-61.
119. Wang, H. Y., D. H. Lee, M. R. D'Andrea, P. A. Peterson, R. P. Shank, and A. B. Reitz, beta-Amyloid(1-42) binds to alpha7 nicotinic acetylcholine receptor with high affinity. Implications for Alzheimer's disease pathology. J Biol Chem, 2000. 275(8): p. 5626-32.
120. Dougherty, J. J., J. Wu, and R. A. Nichols, Beta-amyloid regulation of presynaptic nicotinic receptors in rat hippocampus and neocortex. J Neurosci, 2003. 23(17): p. 6740-7.
121. Nichols, R. A. Beta amyloid regulation of presynaptic calcium in mouse cortex via alpha7-beta2 subtype nicotinic receptors. in Alzheimer's Association. 10th International Conference on Alzheimer's Disease and Related Disorders. 2006. Madrid, Spain.
122. Nayak, S. V., J. J. Dougherty, J. M. McIntosh, and R. A. Nichols, Ca(2+) changes induced by different presynaptic nicotinic receptors in separate populations of individual striatal nerve terminals. J Neurochem, 2001. 76(6): p. 1860-70.
123. Girod, R., N. Barazangi, D. McGehee, and L. W. Role, Facilitation of glutamatergic neurotransmission by presynaptic nicotinic acetylcholine receptors. Neuropharmacology, 2000. 39(13): p. 2715-25.
Ambrogi Lorenzini, C. G., Baldi, E., Bucherelli, C., Sacchetti, B., and Tassoni, G. (1999). Neural topography and chronology of memory consolidation: a review of functional inactivation findings. Neurobiol Learn Mem 71, 1-18.
Arancio, O., Korn, H., Gulyas, A., Freund, T., and Miles, R. (1994). Excitatory synaptic connections onto rat hippocampal inhibitory cells may involve a single transmitter release site. J Physiol 481 (Pt 2), 395-405.
Bliss, T. V., and Collingridge, G. L. (1993). A synaptic model of memory: long-term potentiation in the hippocampus. Nature 361, 31-39.
Cirrito, J. R., May, P. C., O'Dell, M. A., Taylor, J. W., Parsadanian, M., Cramer, J. W., Audia, J. E., Nissen, J. S., Bales, K. R., Paul, S. M., et al. (2003). In vivo assessment of brain interstitial fluid with microdialysis reveals plaque-associated changes in amyloid-beta metabolism and half-life. J Neurosci 23, 8844-8853.
Cirrito, J. R., Yamada, K. A., Finn, M. B., Sloviter, R. S., Bales, K. R., May, P. C., Schoepp, D. D., Paul, S. M., Mennerick, S., and Holtzman, D. M. (2005). Synaptic activity regulates interstitial fluid amyloid-beta levels in vivo. Neuron 48, 913-922.
Cullen, W. K., Suh, Y. H., Anwyl, R., and Rowan, M. J. (1997). Block of LTP in rat hippocampus in vivo by beta-amyloid precursor protein fragments. Neuroreport 8, 3213-3217.
Dawson, G. R., Seabrook, G. R., Zheng, H., Smith, D. W., Graham, S., O'Dowd, G., Bowery, B. J., Boyce, S., Trumbauer, M. E., Chen, H. Y., et al. (1999). Age-related cognitive deficits, impaired long-term potentiation and reduction in synaptic marker density in mice lacking the beta-amyloid precursor protein. Neuroscience 90, 1-13.
Dougherty, J. J., Wu, J., and Nichols, R. A. (2003). Beta-amyloid regulation of presynaptic nicotinic receptors in rat hippocampus and neocortex. J Neurosci 23, 6740-6747.
Garcia-Osta, A., Tsokas, P., Pollonini, G., Landau, E. M., Blitzer, R., and Alberini, C. M. (2006). MuSK expressed in the brain mediates cholinergic responses, synaptic plasticity, and memory formation. J Neurosci 26, 7919-7932.
Genzen, J. R., and McGehee, D. S. (2003). Short- and long-term enhancement of excitatory transmission in the spinal cord dorsal horn by nicotinic acetylcholine receptors. Proceedings of the National Academy of Sciences of the United States of America 100, 6807-6812.
Girod, R., Barazangi, N., McGehee, D., and Role, L. W. (2000). Facilitation of glutamatergic neurotransmission by presynaptic nicotinic acetylcholine receptors. Neuropharmacology 39, 2715-2725.
Gong, B., Vitolo, O. V., Trinchese, F., Liu, S., Shelanski, M., and Arancio, O. (2004). Persistent improvement in synaptic and cognitive functions in an Alzheimer mouse model following rolipram treatment. J Clin Invest 114, 1624-1634.
Gray, R., Rajan, A. S., Radcliffe, K. A., Yakehiro, M., and Dani, J. A. (1996). Hippocampal synaptic transmission enhanced by low concentrations of nicotine. Nature 383, 713-716.
Itoh, A., Akaike, T., Sokabe, M., Nitta, A., Lida, R., Olariu, A., Yamada, K., and Nabeshima, T. (1999). Impairments of long-term potentiation in hippocampal slices of beta-amyloid-infused rats. Eur J Pharmacol 382, 167-175.
Izquierdo, I., and Medina, J. H. (1997). Memory formation: the sequence of biochemical events in the hippocampus and its connection to activity in other brain structures. Neurobiol Learn Mem 68, 285-316.
Ji, D., Lape, R., and Dani, J. A. (2001) Timing and location of nicotinic activity enhances or depresses hippocampal synaptic plasticity. Neuron 31, 131-141.
Jones, S., Sudweeks, S., and Yakel, J. L. (1999). Nicotinic receptors in the brain: correlating physiology with function. Trends Neurosci 22, 555-561.
Klyubin, I., Walsh, D. M., Lernere, C. A., Cullen, W. K., Shankar, G. M., Betts, V., Spooner, E. T., Jiang, L., Anwyl, R., Selkoe, D. J., et al. (2005). Amyloid beta protein immunotherapy neutralizes Abeta oligomers that disrupt synaptic plasticity in vivo. Nature medicine 11, 556-561.
Levin, E. D., and Simon, B. B. (1998). Nicotinic acetylcholine involvement in cognitive function in animals. Psychopharmacology (Berl) 138, 217-230.
Malin, D. H., Crothers, M. K., Lake, J. R., Goyarzu, P., Plotner, R. E., Garcia, S. A., Spell, S. H., Tomsic, B. J., Giordano, T., and Kowall, N. W. (2001). Hippocampal injections of amyloid beta-peptide 1-40 impair subsequent one-trial/day reward learning. Neurobiol Learn Mem 76, 125-137.
Masliah, E. (1995). Mechanisms of synaptic dysfunction in Alzheimer's disease. Histol Histopathol 10, 509-519.
Masliah, E. (2000). The role of synaptic proteins in Alzheimer's disease. Annals of the New York Academy of Sciences 924, 68-75.
Mastrangelo, P., Mathews, P. M., Chishti, M. A., Schmidt, S. D., Gu, Y., Yang, J., Mazzella, M. J., Coomaraswamy, J., Home, P., Strome, B., et al. (2005). Dissociated phenotypes in presenilin transgenic mice define functionally distinct gamma-secretases. Proceedings of the National Academy of Sciences of the United States of America 102, 8972-8977.
Mathews, P. M., Cataldo, A. M., Kao, B. H., Rudnicki, A. G., Qin, X., Yang, J. L., Jiang, Y., Picciano, M., Hulette, C., Lippa, C. F., et al. (2000). Brain expression of presenilins in sporadic and early-onset, familial Alzheimer's disease. Mol Med 6, 878-891.
McGaugh, J. L. (2002). Memory consolidation and the amygdala: a systems perspective. Trends Neurosci 25, 456.
Nichols, R. A. (2006). Beta amyloid regulation of presynaptic calcium in mouse cortex via alpha7-beta2 subtype nicotinic receptors. Paper presented at: Alzheimer's Association 10th International Conference on Alzheimer's Disease and Related Disorders (Madrid, Spain).
Orr-Urtreger, A., Goldner, F. M., Saeki, M., Lorenzo, I., Goldberg, L., De Biasi, M., Dani, J. A., Patrick, J. W., and Beaudet, A. L. (1997). Mice deficient in the alpha7 neuronal nicotinic acetylcholine receptor lack alpha-bungarotoxin binding sites and hippocampal fast nicotinic currents. J Neurosci 17, 9165-9171.
Pawlik, M., Sastre, M., Calero, M., Mathews, P. M., Schmidt, S. D., Nixon, R. A., and Levy, E. (2004). Overexpression of human cystatin C in transgenic mice does not affect levels of endogenous brain amyloid Beta Peptide. J Mol Neurosci 22, 13-18.
Paxinos, G. (1998). Mouse brain in stereotaxic coordinates., 2nd edn (New York, Academic Press).
Phillips, R. G., and LeDoux, J. E. (1992). Differential contribution of amygdala and hippocampus to cued and contextual fear conditioning. Behav Neurosci 106, 274-285.
Phinney, A. L., Calhoun, M. E., Wolfer, D. P., Lipp, H. P., Zheng, H., and Jucker, M. (1999). No hippocampal neuron or synaptic bouton loss in learning-impaired aged beta-amyloid precursor protein-null mice. Neuroscience 90, 1207-1216.
Phinney, A. L., Drisaldi, B., Schmidt, S. D., Lugowski, S., Coronado, V., Liang, Y., Horne, P., Yang, J., Sekoulidis, J., Coomaraswamy, J., et al. (2003). In vivo reduction of amyloid-beta by a mutant copper transporter. Proceedings of the National Academy of Sciences of the United States of America 100, 14193-14198.
Puzzo, D., Vitolo, O., Trinchese, F., Jacob, J. P., Palmeri, A., and Arancio, O. (2005). Amyloid-beta peptide inhibits activation of the nitric oxide/cGMP/cAMP-responsive element-binding protein pathway during hippocampal synaptic plasticity. J Neurosci 25, 6887-6897.
Radcliffe, K. A., and Dani, J. A. (1998). Nicotinic stimulation produces multiple forms of increased glutamatergic synaptic transmission. J Neurosci 18, 7075-7083.
Rozmahel, R., Huang, J., Chen, F., Liang, Y., Nguyen, V., Ikeda, M., Levesque, G., Yu, G., Nishimura, M., Mathews, P., et al. (2002a). Normal brain development in PS1 hypomorphic mice with markedly reduced gamma-secretase cleavage of betaAPP. Neurobiol Aging 23, 187-194.
Rozmahel, R., Mount, H. T., Chen, F., Nguyen, V., Huang, J., Erdebil, S., Liauw, J., Yu, G., Hasegawa, H., Gu, Y., et al. (2002b). Alleles at the Nicastrin locus modify presenilin 1-deficiency phenotype. Proceedings of the National Academy of Sciences of the United States of America 99, 14452-14457.
Schmidt, S. D., Jiang, Y., Nixon, R. A., and Mathews, P. M. (2005a). Tissue processing prior to protein analysis and amyloid-beta quantitation. Methods Mol Biol 299, 267-278.
Schmidt, S. D., Nixon, R. A., and Mathews, P. M. (2005b). ELISA method for measurement of amyloid-beta levels. Methods Mol Biol 299, 279-297.
Seabrook, G. R., Smith, D. W., Bowery, B. J., Easter, A., Reynolds, T., Fitzjohn, S. M., Morton, R. A., Zheng, H., Dawson, G. R., Sirinathsinghji, D. J., et al. (1999). Mechanisms contributing to the deficits in hippocampal synaptic plasticity in mice lacking amyloid precursor protein. Neuropharmacology 38, 349-359.
Selkoe, D. J. (2002). Alzheimer's disease is a synaptic failure. Science 298, 789-791.
Small, D. H., Maksel, D., Kerr, M. L., Ng, J., Hou, X., Chu, C., Mehrani, H., Unabia, S., Azari, M. F., Loiacono, R., et al. (2007). The beta-amyloid protein of Alzheimer's disease binds to membrane lipids but does not bind to the alpha7 nicotinic acetylcholine receptor. Journal of neurochemistry 101, 1527-1538.
Taubenfeld, S. M., Milekic, M. H., Monti, B., and Alberini, C. M. (2001). The consolidation of new but not reactivated memory requires hippocampal C/EBPbeta. Nat Neurosci 4, 813-818.
Vitolo, O. V., Sant'Angelo, A., Costanzo, V., Battaglia, F., Arancio, O., and Shelanski, M. (2002). Amyloid beta-peptide inhibition of the PKA/CREB pathway and long-term potentiation: reversibility by drugs that enhance cAMP signaling. Proceedings of the National Academy of Sciences of the United States of America 99, 13217-13221.
Walsh, D. M., Klyubin, I., Fadeeva, J. V., Cullen, W. K., Anwyl, R., Wolfe, M. S., Rowan, M. J., and Selkoe, D. J. (2002). Naturally secreted oligomers of amyloid beta protein potently inhibit hippocampal long-term potentiation in vivo. Nature 416, 535-539.
Wang, H. Y., Lee, D. H., D'Andrea, M. R., Peterson, P. A., Shank, R. P., and Reitz, A. B. (2000). beta-Amyloid(1-42) binds to alpha7 nicotinic acetylcholine receptor with high affinity. Implications for Alzheimer's disease pathology. The Journal of biological chemistry 275, 5626-5632.
Yao, J., Petanceska, S. S., Montine, T. J., Holtzman, D. M., Schmidt, S. D., Parker, C. A., Callahan, M. J., Lipinski, W. J., Bisgaier, C. L., Turner, B. A., et al. (2004). Aging, gender and APOE isotype modulate metabolism of Alzheimer's Abeta peptides and F-isoprostanes in the absence of detectable amyloid deposits. Journal of neurochemistry 90, 1011-1018.
Zucker, R. S. (1989). Short-term synaptic plasticity. Annu Rev Neurosci 12, 13-31.
Zucker, R. S., and Regehr, W. G. (2002). Short-term synaptic plasticity. Annu Rev Physiol 64, 355-405.

What is claimed is:

1. A method for enhancing memory of a subject, the method comprising administering to the subject an amount of a beta amyloid peptide wherein the amount of amyloid beta peptide administered is sufficient to achieve a concentration of about 200 pM in the hippocampal tissue of the subject.

2. A method for enhancing synaptic plasticity in neurons of a subject, the method comprising administering to the subject a low dose of a beta amyloid peptide.

3. The method of claim 1 or 2, wherein the amyloid beta peptide is Aβ42 having SEQ ID NO: 42.

4. The method of claim 1 or 2, wherein the amyloid beta peptide is a peptide with at least about 75% identity to SEQ ID NO:1, or at least about 80% identity to SEQ ID NO:1, or at least about 85% identity to SEQ ID NO:1, or at least about 90% identity to SEQ ID NO:1, or at least about 95% identity to SEQ ID NO:1, or at least about 97% identity to SEQ ID NO:1, or at least about 99% identity to SEQ ID NO:1.

5. The method of claim 1 or 2, wherein the amount of beta amyloid peptide in the brain following administration is from about 125 pM to about 500 pM, or from about 130 pM to about 480 pM, or from about 140 pM to about 475 pM, or from about 150 pM to about 450 pM, or from about 160 pM to about 440 pM, or from about 170 pM to about 430 pM, or from about 180 pM to about 420 pM, or from about 190 pM to about 410 pM, or from about 200 pM to about 400 pM, or from about 210 pM to about 350 pM, or from about 200 pM to about 300 pM, or from about 200 pM to about 225 pM, or from about 200 pM to about 250 pM, or from about 200 pM to about 275 pM.

6. The method of claim 1 or 2, wherein the subject is suffering from Alzheimer's Disease, head trauma, or an attention deficit disorder.

7. The method of claim 1 or 2, wherein the subject is suffering from a memory disorder.

8. The method of claim 7, wherein the memory disorder comprises or is associated with Alzheimer's disease, Parkinson's disease, Pick's disease, a Lewy body disease, amyotrophic lateral sclerosis, Huntington's disease, Creutzfeld-Jakob disease, Down syndrome, multiple system atrophy, neuronal degeneration with brain iron accumulation type I (Hallervorden-Spatz disease), pure autonomic failure, REM sleep behavior disorder, mild cognitive impairment (MCI), cerebral amyloid angiopathy (CAA), vascular dementias mixed with Alzheimer's disease, aging, a neurodegenerative disease characterized by abnormal amyloid deposition, or any combination thereof.

9. The method of claim 1 or 2, wherein the amyloid beta peptide is administered to the brain of the subject via intralesional, intraperitoneal, intramuscular or intravenous injection; by infusion; by liposome-mediated delivery; or topical, nasal, oral, anal, ocular or otic delivery, or any combination thereof.

10. The method of claim 1 or 2, wherein the amyloid beta peptide is a peptidomimetic.


SEQUENCE LISTING (31 sequences)

SEQ ID NO: 1 - protein, 42 aa, artificial [sequence omitted]

SEQ ID NO: 2 - protein, 42 aa, artificial
  Amyloid-beta (1-42) of Mus musculus; Rattus norvegicus; Rattus rattus; Cricetulus griseus
DAEFGHDSGFEVRHQKLVFFAEDVGSNKGAIIGLMVGGVVIA

SEQ ID NO: 3 - protein, 42 aa, Antechinus stuartii
DAEFRHDSGYEVHHQKLVFFAEDMSSNKGAIIGLMVGGIVIA

SEQ ID NO: 4 - protein, 42 aa, artificial
  Amyloid-beta (1-42)  Xenopus laevis; African clawed frog
DSEYRHDTAYEVHHQKLVFFAEEVGSNKGAIIGLMVGGVVIA

SEQ ID NO: 5 - protein, 7 aa, artificial
  central sequence of amyloid beta (1-42), consensus with Seq IDs 1, 2, 3  and 4
KLVFFAE

SEQ ID NO: 6 - protein, 11 aa, artificial
  Amyloid-beta (1-11), consensus with Seq ID 1, 3
DAEFRHDSGYE

SEQ ID NO: 7 - protein, 28 aa, artificial
  Amyloid-beta (1-28), consensus with Seq ID 1
DAEFRHDSGYEVHHQKLVFFAEDVGSNK

SEQ ID NO: 8 - protein, 38 aa, artificial
  Amyloid-beta (1-38), consensus with Seq ID 1
DAEFRHDSGYEVHHQKLVFFAEDVGSNKGAIIGLMVGG

SEQ ID NO: 9 - protein, 38 aa, artificial; identical to SEQ ID NO: 8
  Amyloid-beta (1-40), consensus with Seq ID 1

SEQ ID NO: 10 - protein, 11 aa, artificial
  Amyloid-beta (10-20), consensus with Seq ID 1, 3, 4
YEVHHQKLVFF

SEQ ID NO: 11 - protein, 17 aa, artificial
  Amyloid-beta (12-28), consensus with Seq ID 1, 4
VHHQKLVFFAEDVGSNK

SEQ ID NO: 12 - protein, 14 aa, artificial
  Amyloid-beta (22-35), consensus with Seq ID 1, 2, 4
EDVGSNKGAIIGLM

SEQ ID NO: 13 - protein, 11 aa, artificial
  Amyloid-beta (25-35), consensus with Seq ID 1, 2, 4
GSNKGAIIGLM

SEQ ID NO: 14 - protein, 5 aa, artificial
  Amyloid-beta (31-35), consensus with Seq ID 1, 2, 3, 4
IIGLM

SEQ ID NO: 15 - protein, 4 aa, artificial
  Amyloid-beta (32-35), consensus with Seq ID 1, 2, 3, 4
IGLM

SEQ ID NO: 16 - protein, 33 aa, artificial
  Amyloid-beta BRI Peptide (1-34)
ASNCFAIRHFENKFAVETLICSRTVKKNIIEEN

SEQ ID NO: 17 - protein, 40 aa, artificial
  Amyloid-beta (1-40) , amide, consensus with Seq ID 1, 9
DAEFRHDSGYEVHHQKLVFFAEDVGSNKGAIIGLMVGGVV

SEQ ID NO: 18 - protein, 43 aa, artificial
  Amyloid-beta (1-43), consensus with Seq ID 1, 9
DAEFRHDSGYEVHHQKLVFFAEDVGSNKGAIIGLMVGGVVIAT

SEQ ID NO: 19 - protein, 12 aa, artificial
  Amyloid-beta (17-28), consensus with Seq ID 1, 2, 4
LVFFAEDVGSNK

SEQ ID NO: 20 - protein, 10 aa, artificial
  Amyloid-beta (33-42), consensus with Seq ID 1, 2, 4
GLMVGGVVIA

SEQ ID NO: 21 - protein, 39 aa, artificial
  Amyloid-beta (3-42), consensus with Seq ID 1
FRHDSGYEVHHQKLVFFAEDVGSNKGAIIGLMVGGVVIA

SEQ ID NO: 22 - protein, 23 aa, artificial
  Amyloid BRI Protein (1-23)
EASNCFAIRHFENKFAVETLICS

SEQ ID NO: 23 - protein, 33 aa, artificial; identical to SEQ ID NO: 16
  Amyloid BRI Protein (1-34), reduced form

SEQ ID NO: 24 - protein, 33 aa, artificial
  Amyloid Dan Protein (1-34)
ASNCFAIRHFENKFAVETLICFNLFLNSQEKHY

SEQ ID NO: 25 - protein, 33 aa, artificial; identical to SEQ ID NO: 24
  Amyloid Dan Protein (1-34), reduced form of Seq ID 24

SEQ ID NO: 26 - protein, 26 aa, artificial
  Amyloid-beta (10-35), amide, consensus with Seq ID 1,  4
YEVHHQKLVFFAEDVGSNKGAIIGLM

SEQ ID NO: 27 - protein, 24 aa, artificial
  Amyloid-beta (17-40), consensus with Seq ID 1, 4
LVFFAEDVGSNKGAIIGLMVGGVV

SEQ ID NO: 28 - protein, 18 aa, artificial
  human Prion Protein (118-135)
AGAVVGGLGGYMLGSAMS

SEQ ID NO: 29 - DNA, 27 bp, artificial
  primer, forward
cctggtcctgctgtgttaaactgcttc

SEQ ID NO: 30 - DNA, 28 bp, artificial
  primer, reverse wt alpha7nAChR-KO plus
ctgctgggaaatcctaggcacacttgag

SEQ ID NO: 31 - DNA, 20 bp, artificial
  primer, reverse neo alpha7nAChR-KO minus
gacaagaccggcttccatcc